(19) United States
(12) Patent Application Publication
(10) Pub. No.: US 2011/0054017 A1
(43) Pub. Date: Mar. 3, 2011

(54) SUBSTITUTED FURANS AND THEIR USE

(76) Inventors: Thomas Lampe, Dusseldorf (DE); Raimund Kast, Wuppertal (DE); Friederike Stoll, Dusseldorf (DE); Joachim Schuhmacher, Wuppertal (DE)

(21) Appl. No.: 12/866,132

(22) PCT Filed: Jan. 29, 2009
(86) PCT No.: PCT/EP09/00553

§ 371 Date: Nov. 15, 2010

(30) Foreign Application Priority Data
Feb. 4, 2008 (DE) 102008007400.4

(51) Int. Cl. A61K 31/341; C07D 307/68; A61P 7/02; A61P 11/00; C07D 307/14; C07D 307/12

(52) U.S. Cl. 514/471; 549/486; 514/461; 549/487; 549/494; 549/501

(57) ABSTRACT

The present application relates to novel substituted furan derivatives, to processes for their preparation, to their use for the treatment and/or prophylaxis of diseases and to their use for preparing medicaments for the treatment and/or prophylaxis of diseases, in particular for the treatment and/or prophylaxis of cardiovascular diseases.

The present application relates to novel substituted furan derivatives, to processes for their preparation, to their use for the treatment and/or prophylaxis of diseases and to their use for preparing medicaments for the treatment and/or prophylaxis of diseases, in particular for the treatment and/or prophylaxis of cardiovascular diseases.

Prostacyclin (PGI₂) belongs to the class of bioactive prostaglandins, which are derivatives of arachidonic acid. PGI₂ is the main product of arachidonic acid metabolism in endothelial cells and is a potent vasodilator and inhibitor of platelet aggregation. PGI₂ is the physiological antagonist of thromboxane A₂ (TxA₂), a strong vasoconstrictor and stimulator of thrombocyte aggregation, and thus contributes to the maintenance of vascular homeostasis. A drop in PGI₂ levels is presumed to be partly responsible for the development of various cardiovascular diseases [Dusting, G. J. et al., Pharmac. Ther. 1990, 48: 323-344; Vane, J. et al., Eur. J. Vasc. Endovasc. Surg. 2003, 26: 571-578].

After release of arachidonic acid from phospholipids via phospholipases A₂, PGI₂ is synthesized by cyclooxygenases and then by PGI₂-synthase. PGI₂ is not stored, but is released immediately after synthesis, exerting its effects locally. PGI₂ is an unstable molecule, which is transformed rapidly (half-life approx. 3 minutes) and non-enzymatically, to an inactive metabolite, 6-keto-prostaglandin-F1alpha [Dusting, G. J. et al., Pharmac. Ther. 1990, 48: 323-344].

The biological effects of PGI₂ occur through binding to a membrane-bound receptor, called the prostacyclin receptor or IP receptor [Narumiya, S. et al., Physiol. Rev. 1999, 79: 1193-1226]. The IP receptor is one of the G-protein-coupled receptors, which are characterized by seven transmembrane domains. In addition to the human IP receptor, prostacyclin receptors have also been cloned from rat and mouse [Vane, J. et al., Eur. J. Vasc. Endovasc. Surg. 2003, 26: 571-578]. In smooth muscle cells, activation of the IP receptor leads to stimulation of adenylate cyclase, which catalyzes the formation of cAMP from ATP. The increase in the intracellular cAMP concentration is responsible for prostacyclin-induced vasodilation and for inhibition of platelet aggregation. In addition to the vasoactive properties, anti-proliferative effects [Schroer, K. et al., Agents Actions Suppl. 1997, 48: 63-91; Kothapalli, D. et al., Mol. Pharmacol. 2003, 64: 249-258; Planchon, P. et al., Life Sci. 1995, 57: 1233-1240] and anti-arteriosclerotic effects [Rudic, R. D. et al., Circ. Res. 2005, 96: 1240-1247; Egan K. M. et al., Science 2004, 114: 784-794] have also been described for PGI₂. Furthermore, PGI₂ also inhibits the formation of metastases [Schneider, M. R. et al., Cancer Metastasis Rev. 1994, 13: 349-64]. It is unclear whether these effects are due to stimulation of cAMP formation or to IP receptor-mediated activation of other signal transduction pathways in the respective target cell [Wise, H. et al. TIPS 1996, 17: 17-21], such as the phosphoinositide cascade, and of potassium channels. Although the effects of PGI₂ are on the whole of benefit therapeutically, clinical application of PGI₂ is severely restricted by its chemical and metabolic instability. PGI₂ analogs that are more stable, for example iloprost [Badesch, D. B. et al., J. Am. Coll. Cardiol. 2004, 43: 56S-61S] and treprostinil [Chattaraj, S. C., Curr. Opion. Invest. Drugs 2002, 3: 582-586] have been made available, but these compounds still have a very short time of action. Moreover, the substances can only be administered to the patient via complicated routes of administration, e.g. by continuous infusion, subcutaneously or via repeated inhalations. These routes of administration can also have additional side-effects, for example infections or pains at the site of injection. The use of beraprost, which to date is the only PGI₂ derivative available for oral administration to the patient [Barst, R. J. et al., J. Am. Coll. Cardiol. 2003, 41: 2119-2125], is once again limited by its short time of action.

The compounds described in the present application are, compared with PGI₂, chemically and metabolically stable, non-prostanoid activators of the IP receptor, which imitate the biological action of PGI₂ and can thus be used for treating diseases, in particular cardiovascular diseases.

U.S. Pat. No. 3,442,913 discloses trifluoromethyl-substituted furancarboxylic acids as synthesis intermediates. EP 0 258 790 describes propargyl furan—and thiophenecarboxylates as insecticides. U.S. Pat. No. 5,068,237 discloses substituted furans for treating Alzheimer's disease, for example. JP 10-114765 claims substituted arylfurans as fungicides. US 2003/0199570 discloses inter alia substituted furans as estrogen receptor modulators for the treatment of chronic inflammatory bowel disease, colitis and Crohn's disease. EP 1 535 915 describes substituted furans and thiophenes as PPAR modulatoren for the treatment of arteriosclerosis, diabetes mellitus and disturbances of lipid metabolism. WO 2004/110357 claims inter alia substituted furans for the treatment of neurodegenerative, cardiovascular and proliferative disorders and eye diseases.

The present invention provides compounds of the general formula (I)

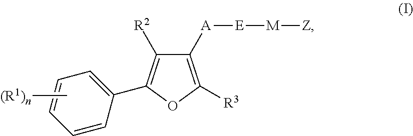

in which

A represents —CH₂— or —C(═O)—,

E represents O or NR⁴,
where
R⁴ represents hydrogen or (C₁-C₄)-alkyl,

M represents a group of the formula

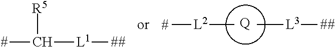

where

# represents the point of attachment to E,

## represents the point of attachment to Z,

R⁵ represents hydrogen or (C₁-C₄)-alkyl,

where alkyl may be substituted by a substituent selected from the group consisting of hydroxyl and amino,

L¹ represents (C₁-C₇)-alkanediyl, (C₂-C₇)-alkenediyl or a group of the formula *-L^{1A}-V-L^{1B}-**,

where alkanediyl and alkenediyl may be substituted by 1 or 2 fluorine substituents,
and
where

* represents the point of attachment to —CHR⁵,

** represents the point of attachment to Z,

L^{1A} represents (C₁-C₅)-alkanediyl,

where alkanediyl may be substituted by 1 or 2 substituents independently of one another selected from the group consisting of (C₁-C₄)-alkyl and (C₁-C₄)-alkoxy,

L^{1B} represents a bond or (C₁-C₃)-alkanediyl,
where alkanediyl may be substituted by 1 or 2 fluorine substituents,
and

V represents O or N—R⁶,
where

R⁶ represents hydrogen, (C₁-C₆)-alkyl or (C₃-C₇)-cycloalkyl,
L² represents a bond or (C₁-C₄)-alkanediyl,

Q represents (C₃-C₇)-cycloalkyl, (C₄-C₇)-cycloalkenyl, 5- to 7-membered heterocyclyl, phenyl or 5- or 6-membered heteroaryl,

where cycloalkyl, cycloalkenyl, heterocyclyl, phenyl and heteroaryl may be substituted by 1 or 2 substituents independently of one another selected from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino, where alkyl may be substituted by a substituent selected from the group consisting of hydroxyl, (C₁-C₄)-alkoxy, amino, mono-(C₁-C₄)-alkylamino and di-(C₁-C₄)-alkylamino, and

L³ represents (C₁-C₄)-alkanediyl or (C₂-C₄)-alkenediyl,

where alkanediyl may be substituted by 1 or 2 fluorine substituents,
and
wherein a methylene group of the alkanediyl group may be replaced by O or N—R⁷,
where

R⁷ represents hydrogen, (C₁-C₆)-alkyl or (C₃-C₇)-cycloalkyl,

Z represents a group of the formula

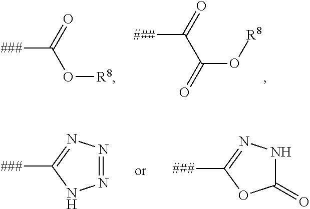

where

### represents the point of attachment to the group L¹ or L³,

and

R⁸ represents hydrogen or (C₁-C₄)-alkyl,

R¹ represents halogen, cyano, nitro, (C₁-C₆)-alkyl, trifluoromethyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethoxy, (C₁-C₆)-alkylthio, (C₁-C₆)-alkylcarbonyl, amino, mono-(C₁-C₆)-alkylamino, di-(C₁-C₆)-alkylamino or (C₁-C₆)-alkylcarbonylamino,

where (C₁-C₆)-alkyl and (C₁-C₆)-alkoxy for their part may be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
or
two radicals R¹ attached to adjacent carbon atoms of the phenyl ring together form a group of the formula —O—CH₂—O—, —O—CHF—O—, —O—CF₂—O—, —O—CH₂—CH₂—O— or —O—CF₂—CF₂—O—,

n represents the number 0, 1 or 2,

where, if R¹ is present more than once, its meaning may in each case be identical or different,
and

R² represents phenyl or 5- or 6-membered heteroaryl,
where phenyl and heteroaryl may be substituted by 1 to 3 substituents independently of one another selected from the group consisting of halogen, cyano, nitro, formyl, (C₁-C₆)-alkyl, trifluoromethyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
   where alkyl and alkoxy may be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
   or
   two substituents attached to adjacent carbon atoms of the phenyl ring together form a group of the formula —O—CH₂—O—, —O—CHF—O—, —O—CF₂—O—, —O—CH₂—CH₂—O— or —O—CF₂—CF₂—O—,
   and

R³ represents methyl, ethyl or trifluoromethyl,

and their salts, solvates and solvates of the salts.

Compounds according to the invention are the compounds of the formula (I) and the salts, solvates and solvates of the salts thereof, the compounds of the formulae below encompassed by the formula (I) and the salts, solvates and solvates of the salts thereof, and also the compounds encompassed by the formula (I) and mentioned below as working examples, and the salts, solvates and solvates of the salts thereof, provided the compounds encompassed by formula (I) and mentioned below are not already salts, solvates and solvates of the salts.

The compounds of the invention may, depending on their structure, exist in stereoisomeric forms (enantiomers, diastereomers). The present invention therefore relates to the enantiomers or diastereomers and respective mixtures thereof. The stereoisomerically pure constituents can be isolated in a known manner from such mixtures of enantiomers and/or diastereomers.

If the compounds of the invention may occur in tautomeric forms, the present invention encompasses all tautomeric forms.

Salts which are preferred for the purposes of the present invention are physiologically acceptable salts of the compounds of the invention. Also encompassed are salts which are themselves unsuitable for pharmaceutical uses but can be used for example for isolating or purifying the compounds of the invention.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, maleic acid, citric acid, fumaric acid, maleic acid and benzoic acid.

Physiologically acceptable salts of the compounds of the invention include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refers for the purposes of the invention to those forms of the compounds of the invention which form, in the solid or liquid state, a complex by coordination with solvent molecules. Hydrates are a specific form of solvates in which the coordination takes place with water. Hydrates are preferred solvates in the context of the present invention.

The present invention additionally encompasses the use of prodrugs of the compounds of the invention. The term “prodrugs” encompasses compounds which themselves may be biologically active or inactive, but are converted during their residence time in the body into compounds of the invention (for example by metabolism or hydrolysis).

In particular, for the compounds of the formula (I) in which

Z represents a group of the formula

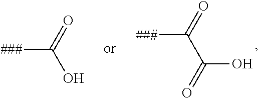

the present invention also includes hydrolyzable ester derivatives of these compounds. These are to be understood as meaning esters which can be hydrolyzed to the free carboxylic acids, as the compounds that are mainly active biologically, in physiologically media, under the conditions of the biological tests described later and in particular in vivo by enzymatic or chemical routes. (C₁-C₄)-alkyl esters, in which the alkyl group can be straight-chain or branched, are preferred as such esters. Particular preference is given to methyl or ethyl esters (see also the corresponding definitions of the radical R⁸).

In the context of the present invention, the substituents have the following meaning, unless specified otherwise:

Alkyl stands in the context of the invention for a straight-chain or branched alkyl radical having 1 to 6 carbon atoms. Preference is given to a straight-chain or branched alkyl radical having 1 to 4 carbon atoms. The following may be mentioned by way of example and by way of preference: methyl, ethyl, n-propyl, isopropyl, n-butyl, isobutyl, sec-butyl, tert-butyl, 1-ethylpropyl, n-pentyl and n-hexyl.

Alkenyl stands in the context of the invention for a straight-chain or branched alkenyl radical having 2 to 6 carbon atoms and one or two double bonds. Preference is given to a straight-chain or branched alkenyl radical having 2 to 4 carbon atoms and one double bond. The following may be mentioned by way of example and by way of preference: vinyl, allyl, isopropenyl and n-but-2-en-1-yl.

Alkynyl stands in the context of the invention for a straight-chain or branched alkynyl radical having 2 to 4 carbon atoms and a triple bond. The following may be mentioned by way of example and by way of preference: ethynyl, n-prop-1-yn-1-yl, n-prop-2-yn-1-yl, n-but-2-yn-1-yl and n-but-3-yn-1-yl.

Alkanediyl stands in the context of the invention for a straight-chain or branched di-valent alkyl radical having 1 to 7 carbon atoms. The following may be mentioned by way of example and by way of preference: methylene, 1,2-ethylene, ethane-1,1-diyl, 1,3-propylene, propane-1,1-diyl, propane-1,2-diyl, propane-2,2-diyl, 1,4-butylene, butane-1,2-diyl, butane-1,3-diyl and butane-2,3-diyl.

Alkenediyl stands in the context of the invention for a straight-chain or branched di-valent alkenyl radical having 2 to 7 carbon atoms and up to 2 double bonds. The following may be mentioned by way of example and by way of preference: ethene-1,1-diyl, ethene-1,2-diyl, propene-1,1-diyl, propene-1,2-diyl, propene-1,3-diyl, but-1-ene-1,4-diyl, but-1-ene-1,3-diyl, but-2-ene-1,4-diyl and buta-1,3-diene-1,4-diyl.

Alkoxy stands in the context of the invention for a straight-chain or branched alkoxy radical having 1 to 6 carbon atoms. Preference is given to a straight-chain or branched alkoxy radical having 1 to 4 carbon atoms. The following may be mentioned by way of example and by way of preference: methoxy, ethoxy, n-propoxy, isopropoxy, n-butoxy, tert-butoxy, n-pentoxy and n-hexoxy.

Alkylthio stands in the context of the invention for a straight-chain or branched alkylthio radical having 1 to 6 carbon atoms. Preference is given to a straight-chain or branched alkylthio radical having 1 to 4 carbon atoms. The following may be mentioned by way of example and by way of preference: methylthio, ethylthio, n-propylthio, isopropylthio, n-butylthio, tert-butylthio, n-pentylthio and n-hexylthio.

Alkylcarbonyl stands in the context of the invention for a straight-chain or branched alkyl radical having 1 to 6 carbon atoms and a carbonyl group attached in position 1.

The following may be mentioned by way of example and by way of preference: methylcarbonyl, ethylcarbonyl, n-propylcarbonyl, isopropylcarbonyl, n-butylcarbonyl, isobutylcarbonyl and tert-butylcarbonyl.

Monoalkylamino stands in the context of the invention for an amino group having a straight-chain or branched alkyl substituent having 1 to 6 carbon atoms. The following may be mentioned by way of example and by way of preference: methylamino, ethylamino, n-propylamino, isopropylamino and tert-butylamino.

Dialkylamino stands in the context of the invention for an amino group having two identical or different straight-chain or branched alkyl substituents having 1 to 6 carbon atoms each. The following may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Alkylcarbonylamino stands in the context of the invention for an amino group which is attached via a carbonyl group to a straight-chain or branched alkyl substituent having 1 to 6 carbon atoms. The following may be mentioned by way of example and by way of preference: methylcarbonylamino, ethylcarbonylamino, n-propylcarbonylamino, isopropylcarbonylamino, n-butylcarbonylamino, isobutylcarbonylamino and tert-butylcarbonylamino.

Cycloalkyl stands in the context of the invention for a monocyclic saturated cycloalkyl group having 3 to 7 carbon atoms. The following may be mentioned by way of example and by way of preference: cyclopropyl, cyclobutyl, cyclopentyl, cyclohexyl and cycloheptyl.

Cycloalkenyl stands in the context of the invention for a monocyclic cycloalkyl group having 4 to 7 carbon atoms and a double bond. The following may be mentioned by way of example and by way of preference: cyclobutenyl, cyclopentenyl, cyclohexenyl and cycloheptenyl.

Heterocyclyl stands in the context of the invention for a saturated monocyclic heterocyclic radical having 5 to 7 ring atoms and up to 3, preferably up to 2, heteroatoms and/or heterogroups from the series N, O, S, SO, SO₂, where a nitrogen atom may also form an N-oxide. Preference is given to 5- or 6-membered saturated heterocyclyl radicals having one or two ring heteroatoms from the series N and O. The following may be mentioned by way of example and by way of preference: pyrrolidinyl, pyrrolinyl, pyrazolidinyl, tetrahydrofuranyl, piperidinyl, piperazinyl, tetrahydropyranyl, morpholinyl, hexahydroazepinyl and hexahydro-1,4-diazepinyl.

Heteroaryl stands in the context of the invention for an aromatic heterocycle (heteroaromatic) having 5 or 6 ring atoms and up to 3 heteroatoms from the series N, O and S, where a nitrogen atom may also form an N-oxide. The following may be mentioned by way of example and by way of preference: furyl, pyrrolyl, thienyl, pyrazolyl, imidazolyl, thiazolyl, oxazolyl, oxadiazolyl, isoxazolyl, isothiazolyl, pyridyl, pyrimidinyl, pyridazinyl and pyrazinyl.

Halogen stands in the context of the invention for fluorine, chlorine, bromine and iodine, preferably for chlorine or fluorine.

If radicals in the compounds according to the invention are substituted, the radicals, unless specified otherwise, may be mono- or polysubstituted. In the context of the present invention, for all radicals that occur more than once, their meanings are independent of one another. Substitution by one, two or three identical or different substituents is preferred. Very particular preference is given to substitution by one substituent.

In the formulae of the group which may represent M or Z, the end point of the line marked by an *, **, #, ##, •, •• or ### label does not represent a carbon atom or a CH₂ group, but is component of the bond to the respective labeled atom to which M or Z is attached.

In the context of the present invention, preference is given to compounds of the formula (I) in which

A represents —CH₂— or —C(═O)—,

E represents O or NR⁴,
where
R⁴ represents hydrogen or (C₁-C₄)-alkyl,

M represents a group of the formula

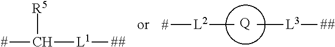

where

# represents the point of attachment to E,

## represents the point of attachment to Z,

R⁵ represents hydrogen, methyl or ethyl,

L¹ represents (C₃-C₇)-alkanediyl, (C₃-C₇)-alkenediyl or a group of the formula *-L^{1A}-V-L^{1B}-**,

where

* represents the point of attachment to —CHR⁵,

** represents the point of attachment to Z,

L^{1A} represents (C₁-C₃)-alkanediyl,

where alkanediyl may be substituted by 1 or 2 substituents independently of one another selected from the group consisting of methyl and ethyl,

L^{1B} represents (C₁-C₃)-alkanediyl,
and

V represents O or N—R⁶,
where

R⁶ represents hydrogen, (C₁-C₃)-alkyl or cyclopropyl,
L² represents a bond, methylene, ethane-1,1-diyl or ethane-1,2-diyl,

Q represents cyclobutyl, cyclopentyl, cyclopentenyl, cyclohexyl, pyrrolidinyl, piperidinyl, tetrahydrofuranyl, tetrahydropyranyl, morpholinyl or phenyl, where cyclobutyl, cyclopentyl, cyclopentenyl, cyclohexyl, pyrrolidinyl, piperidinyl, tetrahydrofuranyl, tetrahydropyranyl, morpholinyl and phenyl may be substituted by 1 or 2 substituents independently of one another selected from the group consisting of fluorine, methyl, ethyl, trifluoromethyl, hydroxyl, methoxy and ethoxy,

and

L³ represents (C₁-C₃)-alkanediyl or a group of the formula •—W—CR⁹R¹⁰—••, •—W—CH₂—CR⁹R¹⁰—•• or •—CH₂—W—CR⁹R¹⁰—••,

where alkanediyl may be substituted by 1 or 2 fluorine substituents,
and
where

• represents the point of attachment to the ring Q,

•• represents the point of attachment to the group Z,

W represents O or N—R⁷,

where

R⁷ represents hydrogen, (C₁-C₃)-alkyl or cyclopropyl,

R⁹ represents hydrogen or fluorine,

and

R¹⁰ represents hydrogen or fluorine,

Z represents a group of the formula

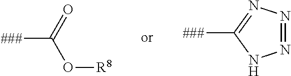

where

### represents the point of attachment to the group L¹ or L³,

and

R⁸ represents hydrogen,

R¹ represents fluorine, chlorine, methyl, ethyl, vinyl, trifluoromethyl or methoxy,

n represents the number 0, 1 or 2,

where, if R¹ is present more than once, its meaning may in each case be identical or different,
and

R² represents phenyl or 2-pyridyl,
where phenyl and 2-pyridyl may be substituted by 1 or 2 substituents independently of one another selected from the group consisting of fluorine, chlorine, cyano, methyl, ethyl, n-propyl, vinyl, methoxy, ethoxy, trifluoromethyl, trifluoromethoxy, methylthio, ethylthio, amino, methylamino and ethylamino,
   and

R³ represents methyl or trifluoromethyl,

and their salts, solvates and solvates of the salts.

In the context of the present invention, particular preference is given to compounds of the formula (I) in which

A represents —CH₂— or —C(═O)—,

E represents O or NR⁴,
where
R⁴ represents hydrogen,

M represents a group of the formula

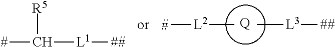

where

# represents the point of attachment to E,

## represents the point of attachment to Z,

R⁵ represents hydrogen or methyl,

L¹ represents butane-1,4-diyl, pentane-1,5-diyl or a group of the formula *-L^{1A}-V-L^{1B}-**,

where

* represents the point of attachment to —CHR⁵,

** represents the point of attachment to Z,

L^{1A} represents methylene or ethane-1,2-diyl,

where methylene and ethane-1,2-diyl may be substituted by 1 or 2 methyl substituents,

L^{1B} represents methylene or ethane-1,2-diyl,
and

V represents O or N—R⁶,
where

R⁶ represents methyl,
L² represents a bond,

Q represents phenyl,

and

L³ represents ethane-1,2-diyl, propane-1,3-diyl or a group of the formula •—W—CR⁹R¹⁰—•• or •—W—CH₂—CR⁹R¹⁰—••,

where

• represents the point of attachment to the ring Q,

•• represents the point of attachment to the group Z,

W represents O,

R⁹ represents hydrogen

and

R¹⁰ represents hydrogen,

Z represents a group of the formula

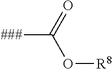

where

### represents the point of attachment to the group L¹ or L³,

and

R⁸ represents hydrogen,

R¹ represents fluorine, chlorine, methyl or trifluoromethyl,

n represents the number 0 or 1,

and

R² represents phenyl,
where phenyl may be substituted by a substituent selected from the group consisting of methyl, ethyl, vinyl, trifluoromethyl, methoxy, ethoxy and trifluoromethoxy,
   and

R³ represents methyl,

and their salts, solvates and solvates of the salts.

Preference is also given to compounds of the formula (I) in which

M represents a group of the formula

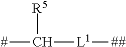

where

# represents the point of attachment to E,

## represents the point of attachment to Z,

R⁵ represents hydrogen or methyl,

L¹ represents butane-1,4-diyl, pentane-1,5-diyl or a group of the formula *-L^{1A}-V-L^{1B}-**,

where

* represents the point of attachment to —CHR⁵,

** represents the point of attachment to Z,

L^{1A} represents methylene or ethane-1,2-diyl,

where methylene and ethane-1,2-diyl may be substituted by 1 or 2 methyl substituents,

L^{1B} represents methylene or ethane-1,2-diyl,
and

V represents O or N—R⁶,
where

R⁶ represents methyl.

Preference is also given to compounds of the formula (I) in which

M represents a group of the formula

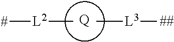

where

# represents the point of attachment to E,

## represents the point of attachment to Z,
L² represents a bond,

Q represents phenyl,

and

L³ represents ethane-1,2-diyl, propane-1,3-diyl or a group of the formula •—W—CR⁹R¹⁰—•• or •—W—CH₂—CR⁹R¹⁰—••,

in which

• represents the point of attachment to the ring Q,

•• represents the point of attachment to the group Z,

W represents O,

R⁹ represents hydrogen,

and

R¹⁰ represents hydrogen.

Preference is also given to compounds of the formula (I) in which

R² represents phenyl,
where phenyl may be substituted by a substituent selected from the group consisting of methyl, ethyl, vinyl, trifluoromethyl, methoxy, ethoxy and trifluoromethoxy.

Preference is also given to compounds of the formula (I) in which R³ represents methyl.

The individual definitions of radicals given in the respective combinations and preferred combinations of radicals are, independently of the given combination of radicals in question, also optionally replaced by radical definitions of other combinations.

Very particular preference is given to combinations of two or more of the preferred ranges mentioned above.

The invention furthermore provides a process for preparing the compounds of the formula (I) according to the invention in which Z represents —COOH, characterized in that either

[A] compounds of the formula (II-A),

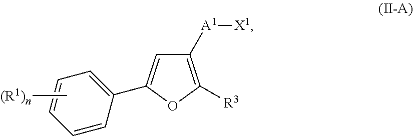

in which n, R¹ and R³ each have the meanings given above
and

A¹ represents —(C═O)—

and

X¹ represents chlorine or hydroxyl,

are coupled in an inert solvent, if appropriate in the presence of a suitable acid or base and/or a suitable condensing agent, with a compound of the formula (III-A)

HE-M-Z¹ (III-A),

in which E and M each have the meanings given above
and

Z¹ represents cyano or a group of the formula COOR^{8A},

where

R^{8A} represents (C₁-C₄)-alkyl,

to give compounds of the formula (IV-A)

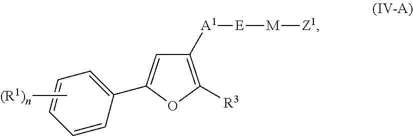

in which n, A¹, E, M, Z¹, R¹ and R³ each have the meanings given above, then brominated in an inert solvent, for example with N-bromosuccinimide, to give compounds of the formula (V-A)

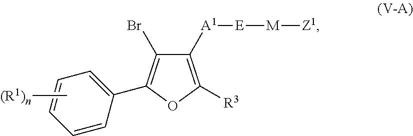

in which n, A¹, E, M, Z¹, R¹ and R³ each have the meanings given above, and these are then coupled in an inert solvent in the presence of a base and a suitable palladium catalyst with a compound of the formula (VI)

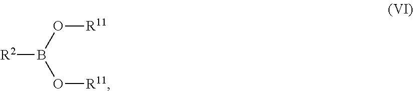

in which R² has the meaning given above,
and

R¹¹ represents hydrogen or both radicals R¹¹ together form a —C(CH₃)₂—C(CH₃)₂— bridge,

to give compounds of the formula (VII-A)

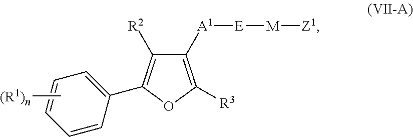

in which n, A¹, E, M, Z¹, R¹, R² and R³ each have the meanings given above,
or

[B] compounds of the formula (II-B)

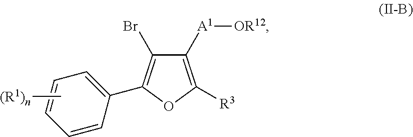

in which n, R¹ and R³ each have the meanings given above
and

A¹ represents —(C═O)—,

and

R¹² represents (C₁-C₄)-alkyl,

are coupled in an inert solvent in the presence of a base and a suitable palladium catalyst with a compound of the formula (VI) to give compounds of the formula (IV-B)

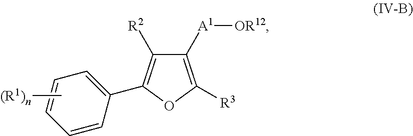

in which n, A¹, R¹, R², R³ and R¹² each have the meanings given above, and these are then converted by basic or acidic hydrolysis into compounds of the formula (V-B)

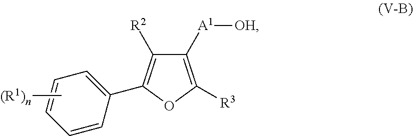

in which n, A¹, R¹, R² and R³ each have the meanings given above, and these are then reacted in an inert solvent in the presence of a suitable base and a suitable condensing agent with a compound of the formula (III-A) to give compounds of the formula (VII-A)

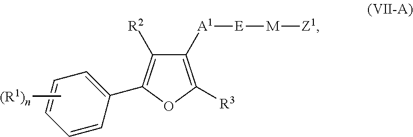

in which n, A¹, E, M, Z¹, R¹, R² and R³ each have the meanings given above,
or

[C] compounds of the formula (II-C)

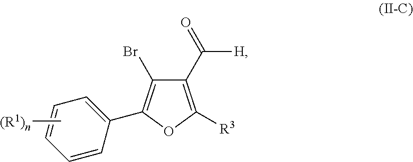

in which n, R¹ and R³ each have the meanings given above,
are coupled in an inert solvent in the presence of a base and a suitable palladium catalyst with a compound of the formula (VI) to give compounds of the formula (IV-C)

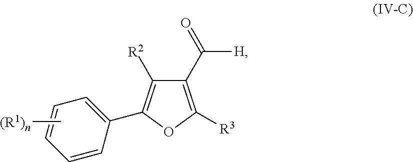

in which n, R¹, R² and R³ each have the meanings given above,
these are then reduced in a suitable solvent with a suitable reducing agent to give compounds of the formula (V-C)

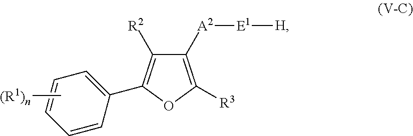

in which n, R¹, R² and R³ each have the meanings given above
and

A² represents —CH₂—

and

E¹ represents O,

and these are then reacted in an inert solvent in the presence of a suitable base with a compound of the formula (III-C)

X²-M-Z¹ (III-C),

in which M and Z¹ each have the meanings given above,
and

X² represents a leaving group, such as, for example, halogen or trifluoromethanesulfonyloxy, in particular bromine or trifluoromethansulfonyloxy,

to give compounds of the formula (VII-C)

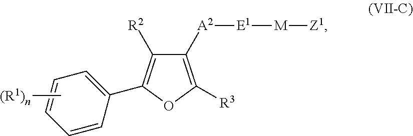

in which n, A², E¹, M, Z¹, R¹, R² and R³ each have the meanings given above,
or

[D] compounds of the formula (IV-C) are reacted in an inert solvent in the presence of a suitable reducing agent with a compound of the formula (III-D)

HE²-M-Z¹ (III-D),

in which M and Z¹ each have the meanings given above,
and

E² represents NR⁴,
where
R⁴ represents hydrogen or (C₁-C₄)-alkyl,
   to give compounds of the formula (VII-D)

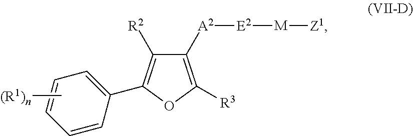

in which n, A², E², M, Z¹, R¹, R² and R³ each have the meanings given above, and the respective compounds of the formulae (VII-A), (VII-C) and (VII-D) obtained are then converted by hydrolysis of the cyano or ester group Z¹ into the carboxylic acids of the formula (I-1)

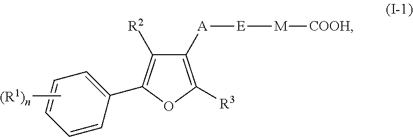

in which n, A, E, M, R¹, R² and R³ each have the meanings given above, and these are, if appropriate, reacted with the appropriate (i) solvents and/or (ii) bases or acids to give their solvates, salts and/or solvates of the salts.

Inert solvents for the coupling reactions (II-A)+(III-A)→(IV-A) and (V-B)+(III-A)→(VII-A) are, for example, ethers, such as diethyl ether, methyl tert-butyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethylene or chlorobenzene, or other solvents, such as acetone, acetonitrile, ethyl acetate, pyridine, dimethyl sulfoxide, dimethylformamide, N,N′-dimethylpropyleneurea (DMPU) or N-methylpyrrolidinone (NMP). It is also possible to use mixtures of the solvents mentioned. Preference is given to dichloromethane, dimethylformamide or mixtures of these two solvents. Suitable bases for the coupling reactions are alkali metal carbonates, for example sodium carbonate or potassium carbonate, or organic bases, such as triethylamine, N-methylmorpholine, N-methylpiperidine, N,N-diisopropylethylamine or 4-N,N-dimethylaminopyridine. Preference is given to using triethylamine.

Acids suitable for the coupling reactions are, in general, sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid. Here, the acid is employed in catalytic amounts.

The couplings (II-A)+(III-A)→(IV-A) and (V-B)+(III-A)→(VII-A) are generally carried out in a temperature range of from 0° C. to +60° C., preferably at from 0° C. to +35° C. The reactions can be carried out at atmospheric, at elevated or at reduced pressure (for example at from 0.5 to 5 bar); they are generally carried out at atmospheric pressure.

Suitable condensing agents for the amide coupling reactions [E=NR⁴ in (III-A)] (II-A)+(III-A)→(IV-A) and (V-B)+(III-A)→(VII-A) are, for example, carbodiimides, such as N,N′-diethyl-, N,N′-dipropyl-, N,N′-diisopropyl-, N,N′-dicyclohexylcarbodiimide (DCC) or N-(3-dimethylaminoisopropyl)-N′-ethylcarbodiimide hydrochloride (EDC), phosgene derivatives, such as N,N′-carbonyldiimidazole (CU), 1,2-oxazolium compounds, such as 2-ethyl-5-phenyl-1,2-oxazolium 3-sulfate or 2-tert-butyl-5-methylisoxazolium perchlorate, acylamino compounds, such as 2-ethoxy-1-ethoxycarbonyl-1,2-dihydroquinoline, or isobutyl chlorformate, propanephosphonic anhydride, diethyl cyanophosphonate, bis-(2-oxo-3-oxazolidinyl)phosphoryl chloride, benzotriazol-1-yloxy-tris(dimethylamino)phosphonium hexafluorophosphate, benzotriazol-1-yloxy-tris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborat (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), if appropriate in combination with further auxiliaries such as 1-hydroxybenzotriazole (HOBt) or N-hydroxysuccinimide (HOSu). Preference is given to using O-(7-azabenzotriazol-1-yl)-N,N,N′,N′-tetramethyluronium hexafluorophosphate (HATU) in combination with N,N-diisopropylethylamine and 4-N,N-dimethylaminopyridine. Suitable inert solvents for the bromination in process step (IV-A)→(V-A) are halogenated hydrocarbons, such as, for example, carbon tetrachloride or 1,2-dichloroethane, or other solvents, such as, for example, acetonitrile. The bromination is carried out in a temperature range of from −20° C. to +50° C. Suitable brominating agents are elemental bromine and in particular N-bromosuccinimide (NBS), if appropriate with addition of α,α′-azobis(isobutyronitrile) (AIBN) as initiator.

Inert solvents for process steps (V-A)+(VI)→(VII-A), (II-B)+(VI)→(IV-B) and (II-C)+(VI)→(IV-C) are, for example, alcohols, such as methanol, ethanol, n-propanol, isopropanol, n-butanol or tert-butanol, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as dimethylformamide, dimethyl sulfoxide, N,N′-dimethylpropyleneurea (DMPU), N-methylpyrrolidone (NMP), pyridine, acetonitrile or else water. It is also possible to use mixtures of the solvents mentioned. Preference is given to a mixture of dimethyl sulfoxide and water.

Suitable bases for the process steps (V-A)+(VI)→(VII-A), (II-B)+(VI)→(IV-B) and (II-C)+(VI)→(IV-C) are customary inorganic bases. These include in particular alkali metal hydroxides, such as, for example, lithium hydroxide, sodium hydroxide or potassium hydroxide, alkali metal bicarbonates, such as sodium bicarbonate or potassium bicarbonate, alkali metal carbonate and alkaline earth metal carbonates, such as lithium carbonate, sodium carbonate, potassium carbonate, calcium carbonate or cesium carbonate, or alkali metal hydrogenphosphates, such as disodium hydrogenphosphate or dipotassium hydrogenphosphate. Preference is given to using sodium carbonate or potassium carbonate.

Suitable palladium catalysts for the process steps (V-A)+(VI)→(VII-A), (II-B)+(VI)→(IV-B) and (II-C)+(VI)→(IV-C) [“Suzuki coupling”] are, for example, palladium on activated carbon, palladium(II) acetate, tetrakis(triphenylphosphine)palladium(0), bis(triphenylphosphine)palladium(II) chloride, bis-(acetonitrile)palladium(II) chloride and [1,1′-bis(diphenylphosphino)ferrocene]dichloropalladium(II)/dichloromethane complex [c.f., for example, J. Hassan et al., Chem. Rev. 102, 1359-1469 (2002)]. The reactions (V-A)+(VI)→(VII-A), (II-B)+(VI)→(IV-B) and (II-C)+(VI)→(IV-C) are generally carried out in a temperature range of from +20° C. to +150° C., preferably at from +50° C. to +100° C.

Suitable inert solvents for the process step (IV-C)→(V-C) are alcohols, such as methanol, ethanol, n-propanol or isopropanol, or ethers, such as diethyl ether, dioxane, tetrahydrofuran, glycol dimethyl ether or diethylene glycol dimethyl ether, or halogenated hydrocarbons, such as dichloromethane, trichloromethane, carbon tetrachloride or 1,2-dichloroethane, or other solvents, such as dimethylformamide. It is also possible to use mixtures of the solvents mentioned. Preference is given to using tetrahydrofuran.

Suitable reducing agents for the process step (IV-C)→(V-C) are borohydrides, such as, for example, sodium borohydride, sodium triacetoxyborohydride, lithium borohydride or sodium cyanoborohydride, aluminum hydrides, such as, for example, lithium aluminum hydride, sodium bis-(2-methoxyethoxy)aluminum hydride or diisobutylaluminum hydride, or borane/tetrahydrofuran complex.

The reaction (IV-C)→(V-C) is generally carried out in a temperature range of from 0° C. to +60° C., preferably from 0° C. to +40° C.

Inert solvents for process step (V-C)+(III-C)→(VII-C) are, for example, ethers, such as diethyl ether, methyl tert-butyl ether, dioxane, tetrahydrofuran, glycol-dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethane, tetrachloroethane, trichloroethylene, chlorobenzene or chlorotoluene, or other solvents, such as dimethylformamide (DMF), dimethyl sulfoxide (DMSO), N,N′-dimethylpropyleneurea (DMPU), N-methylpyrrolidone (NMP) or acetonitrile. It is also possible to use mixtures of the solvents mentioned. Preference is given to using tetrahydrofuran or dimethylformamide.

However, if appropriate, the process step (V-C)+(III-C)→(VII-C) can also be carried out in the absence of a solvent.

Suitable bases for process step (V-C)+(III-C)→(VII-C) are customary inorganic or organic bases. These preferably include alkali metal hydroxides, such as, for example, lithium hydroxide, sodium hydroxide, or potassium hydroxide, alkali metal or alkaline earth metal carbonates, such as lithium carbonate, sodium carbonate, potassium carbonate, calcium carbonate or cesium carbonate, alkali metal alkoxides, such as sodium tert-butoxide or potassium tert-butoxide, alkali metal hydrides, such as sodium hydride or potassium hydride, amides, such as lithium bis(trimethylsilyl)-amide or potassium bis(trimethylsilyl)amide or lithium diisopropylamide, organic metallic compounds, such as butyllithium or phenyllithium, or organic amines, such as triethylamine, N-methylmorpholine, N-methylpiperidine, N,N-diisopropylethylamine or pyridine.

In the case of the reaction (V-C)+(III-C)→(VII-C) phosphazene bases (so-called “Schwesinger bases”), such as, for example, P2-t-Bu or P4-t-Bu are likewise expedient [cf., for example, R. Schwesinger, H. Schlemper, Angew. Chem. Int. Ed. Engl. 26, 1167 (1987); T. Pietzonka, D. Seebach, Chem. Ber. 124, 1837 (1991)]. If appropriate, the process step (V-C)+(III-C)→(VII-C) can advantageously be carried out with addition of a crown ether.

In one process variant, the reactions (V-C)+(III-C)→(VII-C) can also be carried out in a two-phase mixture consisting of an aqueous alkali metal hydroxide solution as base and one of the hydrocarbons or halogenated hydrocarbons mentioned above as further solvent, using a phase-transfer catalyst, such as tetrabutylammonium hydrogen sulfate or tetrabutylammonium bromide.

The process step (V-C)+(III-C)→(VII-C) is generally carried out in a temperature range of from −20° C. to +120° C., preferably at from 0° C. to +60° C.

Suitable inert solvents for process step (IV-C)+(III-D)→(VII-D) are alcohols, such as methanol, ethanol, n-propanol or isopropanol, or ethers, such as diethyl ether, dioxane, tetrahydrofuran, glycol dimethyl ether or diethylene glycol dimethyl ether, or halogenated hydrocarbons, such as dichloromethane, trichloromethane, carbon tetrachloride or 1,2-dichloroethane, or other solvents, such as dimethylformamide. It is also possible to use mixtures of the solvents mentioned. Preference is given to using tetrahydrofuran.

Suitable reducing agents for the process step (IV-C)+(III-D)→(VII-D) are borohydrides, such as, for example, sodium borohydride, sodium triacetoxyborohydride, lithium borohydride or sodium cyanoborohydride, if appropriate with addition of acids, such as formic acid or acetic acid, or Lewis acids, such as titanium(IV) tetrachloride or titanium(IV) isopropoxide carried out. Alternatively, the reaction (IV-C)+(III-D)→(VII-D) can be carried out using ammonium formate or formic acid, or under an atmosphere of hydrogen using catalysts such as Raney nickel, palladium, palladium on activated carbon or platinum.

The reaction (IV-C)+(III-D)→(VII-D) is generally carried out in a temperature range of from 0° C. to +60° C., preferably at from 0° C. to +40° C.

The hydrolysis of the cyano or ester group Z¹ of the compounds (VII-A), (VII-C) or (VII-D) to give compounds of the formula (I-1) and of the esters of the formula (IV-B) to give carboxylic acids of the formula (V-B) is carried out by customary methods by treating the esters or nitriles in inert solvents with acids or bases, where in the latter case the salts initially formed are converted by treatment with acid into the free carboxylic acids. In the case of the tert-butyl esters, the ester cleavage is preferably carried out using acids.

Suitable inert solvents for these reactions are water or the organic solvents customary for ester cleavage. These preferably include alcohols, such as methanol, ethanol, n-propanol, isopropanol, n-butanol or tert-butanol, or ethers, such as diethyl ether, tetrahydrofuran, dioxane or glycol dimethyl ether, or other solvents, such as acetone, dichloromethane, dimethylformamide or dimethyl sulfoxide. It is also possible to use mixtures of the solvents mentioned. In the case of a basic ester hydrolysis, preference is given to using mixtures of water with dioxane, tetrahydrofuran, methanol and/or ethanol, and for nitrile hydrolysis, preference is given to using water and/or n-propanol. In the case of the reaction with trifluoroacetic acid, preference is given to using dichloromethane, and in the case of the reaction with hydrogen chloride, preference is given to using tetrahydrofuran, diethyl ether, dioxane or water.

Suitable bases are the customary inorganic bases. These preferably include alkali metal hydroxides or alkaline earth metal hydroxides, such as, for example, sodium hydroxide, lithium hydroxide, potassium hydroxide or barium hydroxide, or alkali metal carbonates or alkaline earth metal carbonates, such as sodium carbonate, potassium carbonate or calcium carbonate. Particular preference is given to sodium hydroxide or lithium hydroxide.

Acids suitable for the ester cleavage are, in general, sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, if appropriate with added water. Preference is given to hydrogen chloride or trifluoroacetic acid in the case of the tert-butyl esters and to hydrochloric acid in the case of the methyl esters.

The ester cleavage is generally carried out in a temperature range of from 0° C. to +100° C., preferably at from +0° C. to +50° C.

The reactions mentioned can be carried out at atmospheric, elevated or reduced pressure (for example at from 0.5 to 5 bar). In general, the reactions are carried out at atmospheric pressure.

The compounds of the formula (I) according to the invention in which Z represents a group of the formula

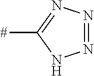

can be prepared by reacting compounds of the formula (VII-A), (VII-C) or (VII-D) in which Z¹ represents cyano in an inert solvent with an alkali metal azide in the presence of ammonium chloride or with trimethylsilyl azide, if appropriate in the presence of a catalyst.

Inert solvents for this reaction are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, or other solvents, such as dimethyl sulfoxide, dimethylformamide, N,N′-dimethylpropyleneurea (DMPU) or N-methylpyrrolidone (NMP). It is also possible to use mixtures of the solvents mentioned. Preference is given to using toluene.

A suitable azide reagent is in particular sodium azide in the presence of ammonium chloride or trimethylsilyl azide. The latter reaction can advantageously be carried out in the presence of a catalyst. Suitable for this purpose are in particular compounds such as di-n-butyltin oxide, trimethylaluminum or zinc bromide. Preference is given to using trimethylsilyl azide in combination with di-n-butyltin oxide.

The reaction is generally carried out in a temperature range of from +50° C. to +150° C., preferably at from +60° C. to +110° C. The reaction can be carried out at atmospheric, elevated or reduced pressure (for example from 0.5 to 5 bar). In general, the reaction is carried out at atmospheric pressure.

The compounds of the formula (I) according to the invention in which Z represents a group of the formula

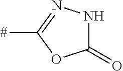

can be prepared by converting compounds of the formula (VII-A), (VII-C) or (VII-D) in which Z¹ represents methoxycarbonyl or ethoxycarbonyl initially in an inert solvent with hydrazine into compounds of the formula (VIII)

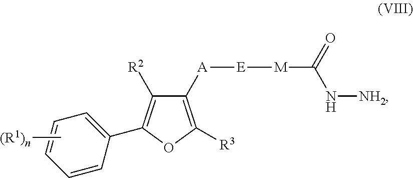

in which n, A, E, M, R¹, R² and R³ have the meanings given above,
and then in an inert solvent with phosgene or a phosgene equivalent, such as, for example, N,N′-carbonyl diimidazole.

Suitable inert solvents for the first step of this reaction sequence are in particular alcohols, such as methanol, ethanol, n-propanol, isopropanol, n-butanol or tert-butanol, or ethers, such as diethyl ether, dioxane, tetrahydrofuran, glycol dimethyl ether or diethylene glycol dimethyl ether. It is also possible to use mixtures of these solvents. Preference is given to using a mixture of methanol and tetrahydrofuran.

The second reaction step is preferably carried out in an ether, in particular in tetrahydrofuran. The reactions are generally carried out in a temperature range of from 0° C. to +70° C., under atmospheric pressure.

The compounds of the formula (I) according to the invention in which L¹ represents a group of the formula *-L^{1A}-V-L^{1B}-** in which L^{1A}, L^{1B} and V have the meanings given above can alternatively also be prepared by converting compounds of the formula (IX)

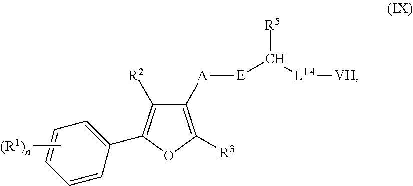

in which n, A, E, L^{1A}, V, R¹, R², R³ and R⁵ each have the meanings given above, in the presence of a base, if appropriate in an inert solvent, with a compound of the formula (X)

X²-L^{1B}-Z¹ (X),

in which L^{1B} and Z¹ have the meanings given above
and
X² represents a leaving group, such as, for example, halogen, mesylate or tosylate,
or, in the case that L^{1B} represents —CH₂CH₂— with a compound of the formula (XI)

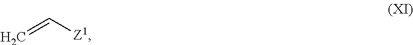

in which Z¹ has the meanings given above,
into compounds of the formula (VII-1)

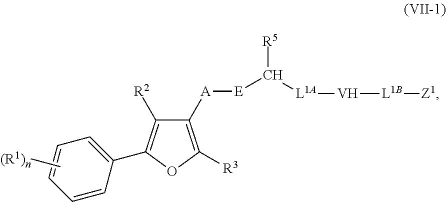

in which n, A, E, L^{1A}, L^{1B}, V, Z¹, R¹, R², R³ and R⁵ each have the meanings given above,
and then reacting these further, in a manner corresponding to the process described above.

For the process steps (IX)+(X) and (XI)→(VII-1), the reaction parameters described above for the reactions (II-A)+(III-A)→(IV-A) and (II-B)+(III-A)→(IV-B), such as solvents, bases and reaction temperatures, are used in an analogous manner.

The compounds of the formula (I) according to the invention in which L³ represents a group of the formula •—W—CR⁹R¹⁰—•• or •—W—CH₂—CR⁹R¹⁰—••, where W, R⁹ and R¹⁰ have the meanings given above, can alternatively also be prepared by converting compounds of the formula (XII)

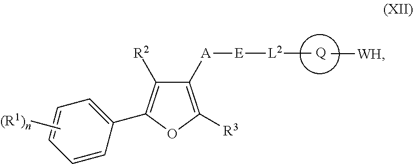

in which n, A, E, L², Q, W, R¹, R² and R³ each have the meanings given above, in the presence of a base, if appropriate in an inert solvent, with a compound of the formula (XIII)

X²—(CH₂)ₘ—CR⁹R¹⁰—Z¹ (XIII),

in which R⁹, R¹⁰, X² and Z¹ each have the meanings given above,

m represents the number 0 or 1,

or, in the case that L³ represents •—W—CH₂CH₂—••, with a compound of the formula (XI)
into compounds of the formula (VII-2)

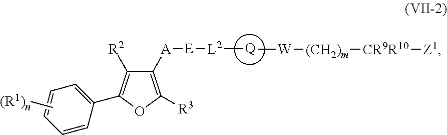

in which n, m, A, E, L², Q, W, Z¹, R¹, R², R³, R⁹, R¹⁰ and m each have the meanings given above,
and then reacting these further, in a manner corresponding to the process described above.

For the process steps (X)+(XIII) and (XI)→(VII-2), the reaction parameters described above for the reactions (II-A)+(III-A)→(IV-A) and (II-B)+(III-A)→(IV-B), such as solvents, bases and reaction temperatures, are used in an analogous manner.

Further compounds according to the invention can optionally also be prepared by conversions of functional groups of individual substituents, in particular those listed under R¹ and R², starting from the compounds of the formula (I) obtained by the above processes. These conversions are carried out by conventional methods known to the person skilled in the art and include, for example, reactions such as nucleophilic or electrophilic substitutions, oxidations, reductions, hydrogenations, transition metal-catalyzed coupling reactions, eliminations, alkylation, amination, esterifications, ester cleavage, etherification, ether cleavage, formation of carboxamides, and also the introduction and removal of temporary protective groups.

The compounds of the formulae (II-A), (II-B), (II-C), (III-A), (III-C), (III-D) and (VI) are commercially available, known from the literature or can be prepared analogously to processes known from the literature (see also reaction schemes 1 and 2).

The preparation of the compounds according to the invention can be illustrated by the synthesis schemes below:

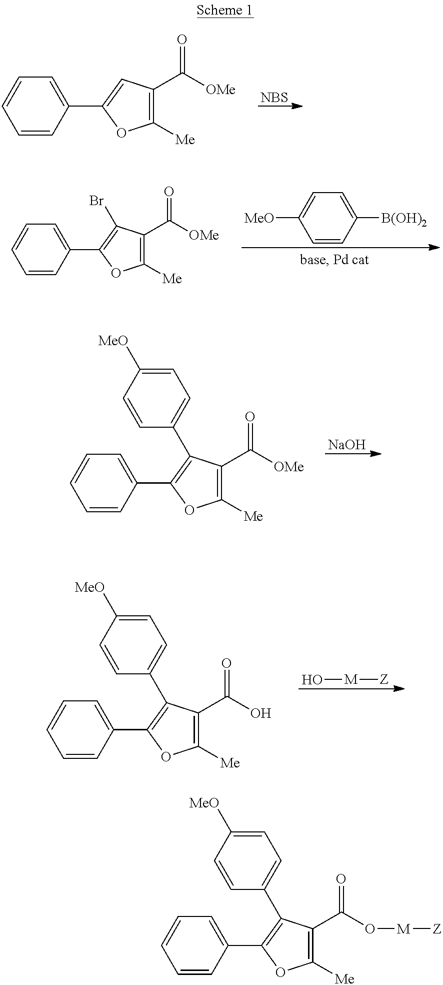

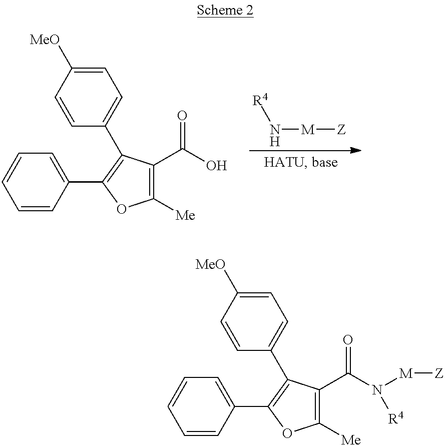

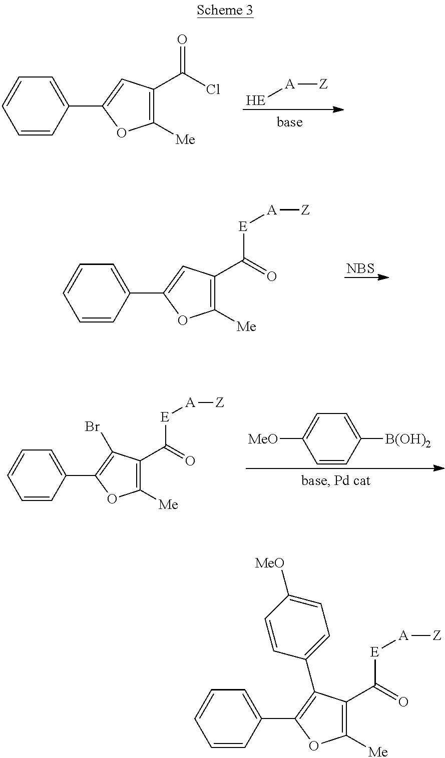

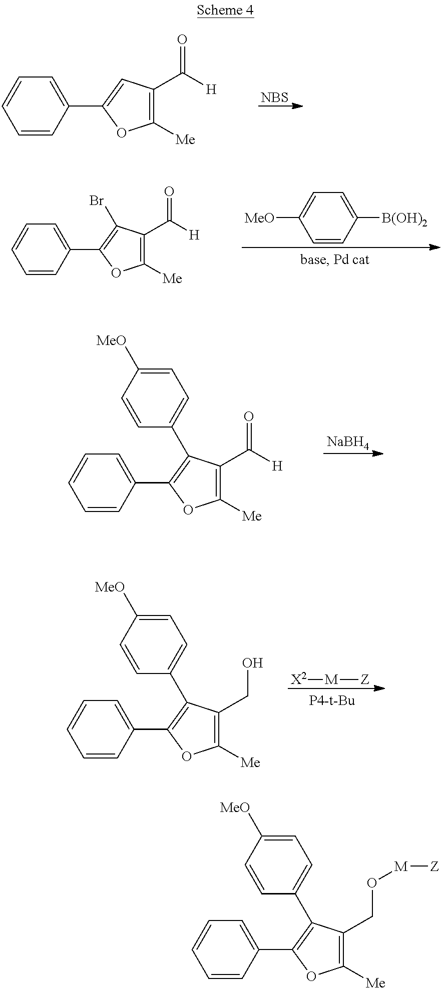

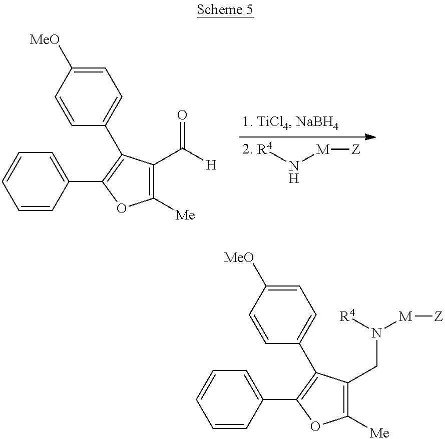

The compounds according to the invention possess valuable pharmacological properties and can be used for the prevention and treatment of diseases in humans and animals. The compounds according to the invention are chemically and metabolically stabile, non-prostanoid activators of the IP receptor.

They are thus suitable in particular for the prophylaxis and/or treatment of cardiovascular diseases such as stable and unstable angina pectoris, of hypertension and heart failure, pulmonary hypertension, for the prophylaxis and/or treatment of thromboembolic diseases and ischemias such as myocardial infarction, stroke, transient and ischaemic attacks and subarachnoid hemorrhage, and for the prevention of restenosis such as after thrombolytic treatments, percutaneous transluminal angioplasty (PTA), coronary angioplasty (PTCA) and bypass surgery.

The compounds according to the invention are particularly suitable for the treatment and/or prophylaxis of pulmonary hypertension (PH) including its various manifestations. The compounds of the invention are therefore particularly suitable for the treatment and/or prophylaxis of pulmonary arterial hypertension (PAH) and its subtypes such as idiopathic and familial pulmonary arterial hypertension, and the pulmonary arterial hypertension which is associated for example with portal hypertension, fibrotic disorders, HIV infection or inappropriate medications or toxins.

The compounds of the invention can also be used for the treatment and/or prophylaxis of other types of pulmonary hypertension. Thus, for example, they can be employed for the treatment and/or prophylaxis of pulmonary hypertension associated with left atrial or left ventricular disorders and with left heart valve disorders. In addition, the compounds of the invention are suitable for the treatment and/or prophylaxis of pulmonary hypertension associated with chronic obstructive pulmonary disease, interstitial pulmonary disease, pulmonary fibrosis, sleep apnoea syndrome, disorders with alveolar hypoventilation, altitude sickness and pulmonary development impairments.

The compounds of the invention are furthermore suitable for the treatment and/or prophylaxis of pulmonary hypertension based on chronic thrombotic and/or embolic disorders such as, for example, thromboembolism of the proximal pulmonary arteries, obstruction of the distal pulmonary arteries and pulmonary embolism. The compounds of the invention can further be used for the treatment and/or prophylaxis of pulmonary hypertension connected with sarcoidosis, histiocytosis X or lymphangioleiomyomatosis, and where the pulmonary hypertension is caused by external compression of vessels (lymph nodes, tumor, fibrosing mediastinitis).

In addition, the compounds according to the invention can also be used for the treatment and/or prophylaxis of peripheral and cardial vascular diseases, peripheral occlusive diseases (PAOD, PVD) and disturbances of peripheral blood flow. Furthermore, the compounds according to the invention can be used for the treatment of arteriosclerosis, hepatitis, asthmatic diseases, chronic obstructive pulmonary diseases (COPD), pulmonary edema, fibrosing lung diseases such as idiopathic pulmonary fibrosis (IPF) and ARDS, inflammatory vascular diseases such as scleroderma and lupus erythematosus, renal failure, arthritis and osteoporosis, and also for the prophylaxis and/or treatment of cancers, especially of metastasizing tumors.

Moreover, the compounds according to the invention can also be used as an addition to the preserving medium of an organ transplant, e.g. kidneys, lungs, heart or islet cells.

The present invention further relates to the use of the compounds according to the invention for the treatment and/or prophylaxis of diseases, and especially of the aforementioned diseases.

The present invention further relates to the use of the compounds according to the invention for the production of a medicinal product for the treatment and/or prophylaxis of diseases, and especially of the aforementioned diseases.

The present invention further relates to a method for the treatment and/or prophylaxis of diseases, especially of the aforementioned diseases, using an effective amount of at least one of the compounds according to the invention.

The present invention further relates to the compounds according to the invention of the formula (I) for use in a method for the treatment and/or prophylaxis of angina pectoris, pulmonary hypertension, thromboembolic disorders and peripheral occlusive diseases.

The compounds of the invention can be employed alone or, if required, in combination with other active ingredients. The present invention further relates to medicaments comprising at least one of the compounds of the invention and one or more further active ingredients, especially for the treatment and/or prophylaxis of the aforementioned disorders. Suitable active ingredients for combinations are by way of example and preferably:

organic nitrates and NO donors such as, for example, sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), such as, for example, inhibitors of phosphodiesterases (PDE) 1, 2, 3, 4 and/or 5, especially PDE 5 inhibitors such as sildenafil, vardenafil and tadalafil;
NO-independent but heme-dependent stimulators of guanylate cyclase such as in particular the compounds described in WO 00/06568, WO 00/06569, WO 02/42301 and WO 03/095451;
NO- and heme-independent activators of guanylate cyclase, such as in particular the compounds described in WO 01/19355, WO 01/19776, WO 01/19778, WO 01/19780, WO 02/070462 and WO 02/070510;
compounds which inhibit human neutrophile elastase (HNE), such as, for example, sivelestat, DX-890 (Reltran), elafin or in particular the compounds described in WO 03/053930, WO 2004/020410, WO 2004/020412, WO 2004/024700, WO 2004/024701, WO 2005/080372, WO 2005/082863 and WO 2005/082864;
compounds which inhibit the signal transduction cascade, for example and preferably from the group of kinase inhibitors, in particular from the group of tyrosine kinase and/or serine/threonine kinase inhibitors;
compounds which inhibit soluble epoxide hydrolase (sEH), such as, for example, N,N′-dicyclohexylurea, 12-(3-adamantan-1-yl-ureido)dodecanoic acid or 1-adamantan-1-yl-3-{5-[2-(2-ethoxyethoxy)ethoxy]pentyl}urea;
compounds which influence the energy metabolism of the heart, such as by way of example and preferably etomoxir, dichloroacetate, ranolazine or trimetazidine;
agonists of VPAC receptors, such as by way of example and preferably the vasocactive intestinal polypeptide;
agents having an antithrombotic effect, for example and preferably from the group of platelet aggregation inhibitors, of anticoagulants or of profibrinolytic substances;
active ingredients which lower blood pressure, for example and preferably from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors and diurectics; and/or
active ingredients which alter lipid metabolism, for example and preferably from the group of thyroid receptor agonists, cholesterol synthesis inhibitors such as by way of example and preferably HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

In a preferred embodiment of the invention, the compounds of the invention are employed in combination with a kinase inhibitor such as by way of example and preferably canertinib, imatinib, gefitinib, erlotinib, lapatinib, lestaurtinib, lonafarnib, pegaptinib, pelitinib, semaxanib, tandutinib, tipifarnib, vatalanib, sorafenib, sunitinib, bortezomib, lonidamin, leflunomid, fasudil or Y-27632.

Agents having an antithrombotic effect preferably mean compounds from the group of platelet aggregation inhibitors, of anticoagulants or of profibrinolytic substances.

In a preferred embodiment of the invention, the compounds of the invention are administered in combination with a platelet aggregation inhibitor such as by way of example and preferably aspirin, clopidogrel, ticlopidine or dipyridamole.

In a preferred embodiment of the invention, the compounds of the invention are administered in combination with a thrombin inhibitor such as by way of example and preferably ximelagatran, melagatran, bivalirudin or clexane.

In a preferred embodiment of the invention, the compounds of the invention are administered in combination with a GPIIb/IIIa antagonist such as by way of example and preferably tirofiban or abciximab.

In a preferred embodiment of the invention, the compounds of the invention are administered in combination with a factor Xa inhibitor such as by way of example and preferably rivaroxaban, DU-176b, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or a low molecular weight (LMW) heparin derivative.

In a preferred embodiment of the invention, the compounds of the invention are administered in combination with a vitamin K antagonist such as by way of example and preferably coumarin.

Agents which lower blood pressure preferably mean compounds from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors, and diuretics.

In a preferred embodiment of the invention, the compounds of the invention are administered in combination with a calcium antagonist such as by way of example and preferably nifedipine, amlodipine, verapamil or diltiazem.

In a preferred embodiment of the invention, the compounds of the invention are administered in combination with an alpha-1 receptor blocker such as by way of example and preferably prazosin.

In a preferred embodiment of the invention, the compounds of the invention are administered in combination with a beta-receptor blocker such as by way of example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin All antagonist such as by way of example and preferably losartan, candesartan, valsartan, telmisartan or embusartan.

In a preferred embodiment of the invention, the compounds of the invention are administered in combination with an ACE inhibitor such as by way of example and preferably enalapril, captopril, lisinopril, ramipril, delapril, fosinopril, quinopril, perindopril or trandopril.

In a preferred embodiment of the invention, the compounds of the invention are administered in combination with an endothelin antagonist such as by way of example and preferably bosentan, darusentan, ambrisentan or sitaxsentan.

In a preferred embodiment of the invention, the compounds of the invention are administered in combination with a renin inhibitor such as by way of example and preferably aliskiren, SPP-600 or SPP-800.

In a preferred embodiment of the invention, the compounds of the invention are administered in combination with a mineralocorticoid receptor antagonist such as by way of example and preferably spironolactone or eplerenone.

In a preferred embodiment of the invention, the compounds of the invention are administered in combination with a Rho kinase inhibitor such as by way of example and preferably fasudil, Y-27632, SLx-2119, BF-66851, BF-66852, BF-66853, KI-23095, SB-772077, GSK-269962A or BA-1049.

In a preferred embodiment of the invention, the compounds of the invention are administered in combination with a diuretic such as by way of example and preferably furosemide.

Agents which alter lipid metabolism preferably mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and lipoprotein(a) antagonists.

In a preferred embodiment of the invention, the compounds of the invention are administered in combination with a CETP inhibitor such as by way of example and preferably torcetrapib (CP-529 414), JJT-705 or CETP vaccine (Avant).

In a preferred embodiment of the invention, the compounds of the invention are administered in combination with a thyroid receptor agonist such as by way of example and preferably D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins such as by way of example and preferably lovastatin, simvastatin, pravastatin, fluvastatin, atorvastatin, rosuvastatin, cerivastatin or pitavastatin.

In a preferred embodiment of the invention, the compounds of the invention are administered in combination with a squalene synthesis inhibitor such as by way of example and preferably BMS-188494 or TAK-475.

In a preferred embodiment of the invention, the compounds of the invention are administered in combination with an ACAT inhibitor such as by way of example and preferably avasimibe, melinamide, pactimibe, eflucimibe or SMP-797.

In a preferred embodiment of the invention, the compounds of the invention are administered in combination with an MTP inhibitor such as by way of example and preferably implitapide, BMS-201038, R-103757 or JTT-130.

In a preferred embodiment of the invention, the compounds of the invention are administered in combination with a PPAR-gamma agonist such as by way of example and preferably pioglitazone or rosiglitazone.

In a preferred embodiment of the invention, the compounds of the invention are administered in combination with a PPAR-delta agonist such as by way of example and preferably GW-501516 or BAY 68-5042.

In a preferred embodiment of the invention, the compounds of the invention are administered in combination with a cholesterol absorption inhibitor such as by way of example and preferably ezetimibe, tiqueside or pamaqueside.

In a preferred embodiment of the invention, the compounds of the invention are administered in combination with a lipase inhibitor such as by way of example and preferably orlistat.

In a preferred embodiment of the invention, the compounds of the invention are administered in combination with a polymeric bile acid adsorbent such as by way of example and preferably cholestyramine, colestipol, colesolvam, CholestaGel or colestimide.

In a preferred embodiment of the invention, the compounds of the invention are administered in combination with a bile acid reabsorption inhibitor such as by way of example and preferably ASBT (═IBAT) inhibitors such as, for example, AZD-7806, S-8921, AK-105, BARI-1741, SC-435 or SC-635.

In a preferred embodiment of the invention, the compounds of the invention are administered in combination with a lipoprotein(a) antagonist such as by way of example and preferably gemcabene calcium (CI-1027) or nicotinic acid.

The present invention further relates to medicaments comprising at least one of the compounds according to the invention, usually in combination with one or more inert, non-toxic, pharmaceutically suitable excipients, and their use for the purposes mentioned above.

The compounds of the invention may have systemic and/or local effects. For this purpose, they can be administered in a suitable way such as, for example, by the oral, parenteral, pulmonary, nasal, sublingual, lingual, buccal, rectal, dermal, transdermal, conjunctival or otic route or as implant or stent.

The compounds of the invention can be administered in administration forms suitable for these administration routes.

Suitable for oral administration are administration forms which function according to the prior art and deliver the compounds of the invention rapidly and/or in a modified manner, and which contain the compounds of the invention in crystalline and/or amorphized and/or dissolved form, such as, for example, tablets (uncoated and coated tablets, for example having coatings which are resistant to gastric juice or are insoluble or dissolve with a delay and control the release of the compound of the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates or sterile powders.

Suitable for the other routes of administration are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears and eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

Oral or parenteral administration are preferred, especially oral and intravenous administration.

The compounds of the invention can be converted into the stated administration forms. This can take place in a manner known per se by mixing with inert, non-toxic, pharmaceutically suitable excipients. These excipients include inter alia carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorings (e.g. inorganic pigments such as, for example, iron oxides) and masking flavors and/or odors.

It has generally proved to be advantageous on parenteral administration to administer amounts of about 0.001 to 1 mg/kg, preferably about 0.01 to 0.5 mg/kg of body weight to achieve effective results. On oral administration, the dosage is about 0.01 to 100 mg/kg, preferably about 0.01 to 20 mg/kg, and very particularly preferably 0.1 to 10 mg/kg of body weight.

It may nevertheless be necessary where appropriate to deviate from the stated amounts, in particular as a function of body weight, administration route, individual response to the active ingredient, type of preparation and time or interval over which administration takes place. Thus, in some cases it may be sufficient to make do with less than the aforementioned minimum amount, whereas in other cases the upper limit mentioned must be exceeded. Where relatively large amounts are administered, it may be advisable to distribute these in a plurality of single doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data of liquid/liquid solutions are, unless indicated otherwise, based in each case on the volume.

### A. EXAMPLES

### Abbreviations

abs. absolute
Ac acetyl
Ac₂O acetic anhydride
Boc tert-butoxycarbonyl
br s broad singulet (in NMR)
Bu butyl
c concentration
DBU 1,8-diazabicyclo[5.4.0]undec-7-ene
DC thin-layer chromatography
DCI direct chemical ionization (in MS)
dd doublet of doublet (in NMR)
DIBAH diisobutylaluminum hydride
DIEA diisopropylethylamine (“Hünig base”)
4-DMAP 4-N,N-dimethylaminopyridine
DME 1,2-dimethoxyethane
DMF N,N-dimethylformamide
DMSO dimethyl sulfoxide
dt doublet of triplet (in NMR)
ee enantiomeric excess
EI electron impact ionization (in MS)
ESI electrospray ionization (in MS)
Et ethyl
m.p. melting point
GC gas chromatography
sat. saturated
h hour(s)
HATU O-(7-azabenzotriazol-1-yl)-N,N,N′,N′-tetramethyluronium hexafluorophosphate
HPLC high-performance liquid chromatography
conc. concentrated
LC-MS liquid chromatography-coupled mass spectrometry
Me methyl
min minute(s)
Ms methanesulfonyl (mesyl)
MS mass spectrometry
NBS N-bromosuccinimide
NMR nuclear magnetic resonance spectrometry
Pd/C palladium on carbon
qu quintet (in NMR)
rac. racemic
RP reverse phase (in HPLC)
RT room temperature
Rₜ retention time (in HPLC)
TFA trifluoroacetic acid
THF tetrahydrofuran

### LC-MS, HPLC and GC Methods:

GC-MS (method 1): instrument: Micromass GCT, GC6890; column: Restek RTX-35, 15 m×200 μm×0.33 μm; constant helium flow: 0.88 ml/min; oven: 70° C.; inlet: 250° C.; gradient: 70° C., 30° C./min→310° C. (maintained for 3 min).

LC-MS (method 2): MS instrument type: Micromass ZQ; HPLC instrument type: Waters Alliance 2795; column: Phenomenex Synergi 2.5μ MAX-RP 100A Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

LC-MS (method 3): MS instrument type: Micromass ZQ; HPLC instrument type: HP 1100 series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

LC-MS (method 4): instrument: Micromass QuattroPremier with Waters UPLC Acquity; column: Thermo Hypersil GOLD 1.9μ 50×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A oven: 50° C.; flow rate: 0.33 ml/min; UV detection: 210 nm.

LC-MS (method 5): instrument: Micromass Quattro LCZ with HPLC Agilent series 1100; column: Phenomenex Synergi 2.5μ MAX-RP 100A Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.1 min 90% A; flow rate: 2 ml/min; oven: 50° C.; UV detection: 208-400 nm.

LC-MS (method 6): MS instrument type: Waters ZQ; HPLC instrument type: Agilent 1100 series; UV DAD; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A→4.1 min 100%; flow rate: 2.5 ml/min; oven: 55° C.; UV detection: 210 nm.

LC-MS (method 7): instrument: Micromass Quattro Micro MS with HPLC Agilent series 1100; column: Thermo Hypersil GOLD 3μ 20×4 mm; mobile phase A: 1 l of water+0.5 ml of 50% strength formic acid, mobile phase B: 1 l of acetonitrile+0.5 ml of 50% strength formic acid; gradient: 0.0 min 100% A 3.0 min 10% A 4.0 min 10% A 4.01 min 100% A (flow rate 2.5 ml/min) 5.00 min 100% A; oven: 50° C.; flow rate: 2 ml/min; UV detection: 210 nm.

### Starting Materials and Intermediates:

### Example 1A

### tert-Butyl (2E,6R)-6-hydroxyhept-2-enoate

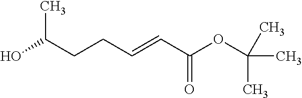

Solution A: 10.71 g (267.7 mmol) of 60% strength sodium hydride are suspended in 150 ml of abs. THF, and 43.3 ml (276.7 mmol) of tert-butyl P,P-dimethylphosphonate are added dropwise with cooling. The mixture is stirred at RT, and after about 30 min a solution is formed.

187.4 ml (187.4 mmol) of a 1 M solution of DIBAH in THF are added dropwise to a solution, cooled to −78° C., of 17.87 g (178.5 mmol) of (R)-γ-valerolactone [(5R)-5-methyldihydrofuran-2(3H)-one] in 200 ml abs. THF. The solution is stirred at −78° C. for another 1 h, and solution A, prepared above, is then added. After the addition has ended, the mixture is slowly warmed to RT and stirred at RT overnight. The reaction mixture is added to 300 ml of ethyl acetate and extracted with 50 ml of concentrated potassium sodium tartrate solution. After phase separation, the aqueous phase is re-extracted with ethyl acetate. The organic phases are combined, washed with saturated aqueous sodium chloride solution, dried over magnesium sulfate and concentrated under reduced pressure. The residue is purified by chromatography on silica gel (mobile phase: cyclohexane/ethyl acetate 5:1). This gives 32.2 g (90.1% of theory) of the target product, which contains small amounts of the cis-isomer.

MS (DCI): m/z=218 (M+NH₄)⁺

¹H-NMR (400 MHz, DMSO-d₆): δ=6.70 (dt, 1H), 5.73 (d, 1H), 4.44 (d, 1H), 3.58 (m, 1H), 2.28-2.13 (m, 2H), 1.47-1.40 (m, 2H), 1.45 (s, 9H), 1.04 (d, 3H).

### Example 2A

### tert-Butyl(−)-6-hydroxyheptanoate

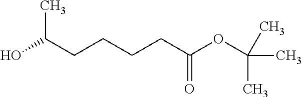

32.2 g (160.8 mmol) of tert-butyl (2E,6R)-6-hydroxyhept-2-enoate are dissolved in 200 ml of ethanol, and 1.7 g of 10% palladium on carbon are added. The mixture is stirred at RT under an atmosphere of hydrogen (atmospheric pressure) for 2 h and then filtered through Celite. The filtrate is concentrated under reduced pressure. The residue gives, after chromatography on silica gel (mobile phase: cyclohexane/ethyl acetate 10:1→6:1), 15.66 g of the target product (48.1% of theory).

MS (DCI): m/z=220 (M+NH₄)⁺

¹H-NMR (400 MHz, CDCl₃): δ=3.85-3.75 (m, 1H), 2.22 (t, 2H), 1.68-1.54 (m, 2H), 1.53-1.30 (m, 4H), 1.45 (s, 9H), 1.18 (d, 3H).

[α]_{D}²⁰=−21°, c=0.118, chloroform.

### Example 3A

### tert-Butyl 6-oxoheptanoate

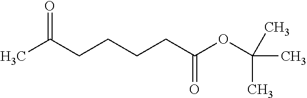

10.0 g (about 90% pure, 62.4 mmol) of 6-oxoheptanoic acid are initially charged in 71.8 ml of cyclohexane, and 20.46 g (93.6 mmol) of tert-butyl 2,2,2-trichloroacetimidate and 15 ml of dichloromethane are added. At −10° C., 0.55 ml (6.24 mmol) of trifluoromethanesulfonic acid is slowly added dropwise to the solution. The resulting suspension is stirred overnight and warmed to RT over this period. The insoluble precipitate is removed by filtration and the filtrate is washed twice with saturated aqueous sodium bicarbonate solution and with saturated aqueous sodium chloride solution, dried over magnesium sulfate and concentrated under reduced pressure. The residue is purified by chromatography on silica gel (cyclohexane/ethyl acetate 5:1). On standing, a solid precipitates from the resulting product overnight. This solid is removed by filtration. This gives 4.51 g of product (36.1% of theory).

GC-MS (method 1): Rₜ=4.1 min; m/z=144 (M-56)⁺

MS (DCI): m/z=218 (M+NH₄)⁺

¹H-NMR (400 MHz, DMSO-d₆): δ=2.46-2.42 (m, 2H), 2.20-2.15 (m, 2H), 2.08 (s, 3H), 1.47-1.40 (m, 4H), 1.41 (s, 9H).

### Example 4A

### tert-Butyl(+/−)-aminoheptanoate

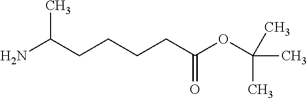

At RT, 3.85 g (49.9 mmol) of ammonium acetate and 345 mg (5.49 mmol) of sodium cyanoborohydride are added to a solution of 1.0 g (4.99 mmol) of tert-butyl 6-oxoheptanoate in 5 ml of methanol. The mixture is stirred at RT overnight and then diluted with water. The aqueous phase is saturated with sodium chloride and extracted three times with dichloromethane. By addition of saturated aqueous sodium carbonate solution, the pH of the aqueous phase is adjusted to 11-12, and the aqueous phase is extracted three times with ethyl acetate. All organic phases are combined, dried over magnesium sulfate and carefully concentrated under reduced pressure. The crude product is purified by chromatography on silica gel (gradient of dichloromethane/isopropanol 20:1 to 3:1, with 1% ammonia). The product fractions are combined and stored at −20° C. This gives 470 mg of product (46.7% of theory).

MS (DCI): m/z=202 (M+H)⁺

¹H-NMR (500 MHz, DMSO-d₆): δ=2.95-2.89 (m, 1H), 2.19 (t, 2H), 1.52-1.43 (m, 2H), 1.41 (s, 9H), 1.35-1.25 (m, 4H), 1.04 (d, 3H).

### Example 5A

### tert-Butyl (6S)-6-bromoheptanoate

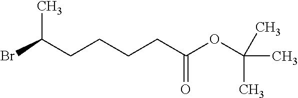

Under argon, 6.0 g (29.6 mmol) of tert-butyl(−)-6-hydroxyheptanoate are dissolved in 60 ml of absolute dichloromethane and, at 0° C., added dropwise over a period of 2 h to a solution of 13.77 g (32.6 mmol) of triphenylphosphine dibromide in 90 ml absolute toluene. After the addition has ended, cooling is removed and the reaction mixture is stirred at RT for 2 h and then filtered off through Celite (the Celite is subsequently washed with dichloromethane). The filtrate is washed with water, dried over sodium sulfate and concentrated under reduced pressure. The crude product is purified by chromatography on silica gel (cyclohexane/ethyl acetate 50:1). This gives 4.79 g of product (60.9% of theory).

MS (DCI): m/z=265/267 (M+H)⁺, 282/284 (M+NH₄)⁺

¹H-NMR (400 MHz, DMSO-d₆): δ=4.28 (m, 1H), 2.21 (t, 2H), 1.80-1.72 (m, 2H), 1.67 (d, 3H), 1.58-1.41 (m, 4H), 1.40 (s, 9H).

[α]_{D}²⁰=+30.0°, c=0.550, chloroform.

### Example 6A

### tert-Butyl (6R)-6-azidoheptanoate

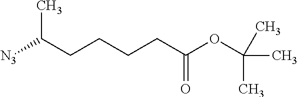

2.0 g (7.45 mmol) of tert-butyl (6S)-6-bromoheptanoate and 2.94 g (45.3 mmol) of sodium azide are mixed in 28.7 ml of DMF and stirred vigorously at 70° C. overnight. After cooling, the reaction mixture is diluted with a large quantity of dichloromethane and washed with saturated aqueous sodium bicarbonate solution and saturated aqueous sodium chloride solution, dried over magnesium sulfate and carefully concentrated under reduced pressure. The crude product is purified by chromatography on silica gel (cyclohexane/ethyl acetate 50:1). This gives 1.63 g of product (95.3% of theory).

MS (DCI): m/z=228 (M+H)⁺, 245 (M+NH₄)⁺

¹H-NMR (400 MHz, DMSO-d₆): δ=3.55 (m, 1H), 2.19 (t, 3H), 1.55-1.25 (m, 6H and s, 9H), 1.18 (d, 3H).

### Example 7A

### tert-Butyl (6R)-6-aminoheptanoate

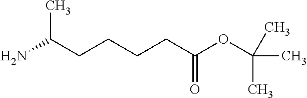

Under argon, 70 mg Pd/C (10%) are added to 1.42 g (6.25 mmol) of tert-butyl (6R)-6-azidoheptanoate dissolved in 200 ml of THF. The mixture is stirred vigorously under an atmosphere of hydrogen at atmospheric pressure and RT overnight. The mixture is filtered through Celite (which is then washed with dichlormethane) and the filtrate is carefully concentrated under reduced pressure. The crude product is purified by chromatography on silica gel (gradient of dichloromethane/isopropanol 20:1 to 3:1, with 1% ammonia). The product fractions are combined and stored at −20° C. This gives 677 mg of product (53.9% of theory).

MS (DCI): m/z=202 (M+H)⁺

¹H-NMR (400 MHz, DMSO-d₆): δ=2.20 (m, 1H), 2.18 (t, 2H), 1.51-1.42 (m, 2H), 1.40 (s, 9H), 1.35-1.27 (m, 4H), 0.94 (d, 3H).

### Example 8A

### Methyl 3-nitrophenoxyacetate

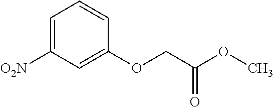

50 g (359.4 mmol) of 3-nitrophenol and 175.67 g (539 mmol) of cesium carbonate are initially charged in 1.01 of acetone, and 71.5 g (467.3 mmol) of methyl bromoacetate are added. The mixture is stirred at 50° C. for 1 h and, after cooling, poured into 7.51 of water. The suspension is stirred for 30 min and then filtered off with suction, and the filter residue is washed with water. The solid is dried in a drying cabinet at 50° C. and 100 mbar. This gives 64.3 g (84.7% of theory) of the target compound.

MS (DCI): m/z=229 (M+NH₄)⁺

¹H-NMR (300 MHz, CDCl₃): δ=7.90 (dd, 1H), 7.43 (t, 1H), 7.48 (t, 1H), 7.28 (dd, 1H), 4.75 (s, 2H), 3.86 (s, 3H).

### Example 9A

### Methyl 3-aminophenoxyacetate

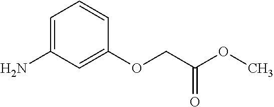

Under argon, 1.3 g of palladium on activated carbon (10%) are added to 13 g (61.6 mmol) of methyl 3-nitrophenoxyacetate in 150 ml of methanol. The mixture is stirred at RT under an atmosphere of hydrogen (atmospheric pressure) for 18 h. The catalyst is filtered off through kieselguhr and the filtrate is concentrated under reduced pressure. This gives, after drying under high vacuum, 10.7 g (95.9% of theory) of the target compound.

MS (DCI): m/z=182 (M+H)⁺, 199 (M+NH₄)⁺

¹H-NMR (400 MHz, CDCl₃): δ=7.10-7.02 (m, 1H), 6.35-6.23 (m, 2H), 4.58 (s, 2H), 3.79 (s, 3H), 3.65 (br s, 2H).

### Example 10A

### 4-Bromo-2-methyl-5-phenylfuran-3-carbaldehyde

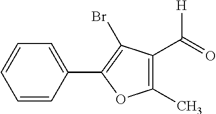

Under argon, 200 mg (1.07 mmol) of 2-methyl-5-phenyl-3-furaldehyde are dissolved in 4 ml of acteonitrile, and five portions of in total 210 mg (1.1.8 mmol) of N-bromosuccinimide are added at −20° C. After 30 min, the reaction solution, which had been diluted with acetonitrile in the meantime, is warmed from −20° C. to RT and concentrated under reduced pressure. The residue is purified by chromatography on silica gel (cyclohexane/ethyl acetate 20:1). This gives 211.6 mg of the target product (74.3% of theory).

GC-MS (method 1): Rₜ=6.57 min; m/z=264/266 (M+H)⁺

¹H-NMR (400 MHz, DMSO-d₆): δ=9.95 (s, 1H), 7.91 (d, 2H), 7.56-7.44 (m, 3H), 2.69 (s, 3H).

### Example 11A

### 4-(4-Methoxyphenyl)-2-methyl-5-phenylfuran-3-carbaldehyde

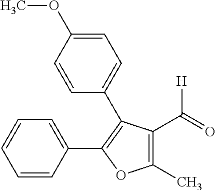

Under argon, 400.0 mg (1.51 mmol) of 4-bromo-2-methyl-5-phenylfuran-3-carbaldehyde, 275.1 mg (1.81 mmol) of 4-methoxyphenylboronic acid and 63.5 mg (0.91 mmol) of bis(triphenylphosphine)palladium(II) chloride are mixed in 1.75 ml of DMSO, and 1.5 ml of 2N aqueous sodium carbonate solution are added. The mixture is stirred vigorously for a total of 4 h at 80° C. (during which time, after about 2 h, a further 92 mg of 4-methoxyboronic acid are added). After cooling, the crude product is separated directly by preparative RP-HPLC (acetonitrile/water gradient), giving 296.4 mg of the target product (67.2% of theory).

LC-MS (method 2): Rₜ=2.38 min; m/z=293 (M+H)⁺

¹H-NMR (500 MHz, DMSO-d₆): δ=9.78 (s, 1H), 7.36-7.25 (m, 6H), 7.01 (d, 2H), 3.79 (s, 3H), 2.69 (s, 3H).

### Example 12A

### [4-(4-Methoxyphenyl)-2-methyl-5-phenylfuran-3-yl]methanol

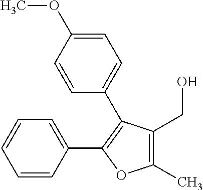

At RT, 22.6 mg (0.6 mmol) of sodium borohydride are added in several portions to a solution of 175 mg (0.60 mmol) of 4-(4-methoxyphenyl)-2-methyl-5-phenylfuran-3-carbaldehyde in 0.7 ml of ethanol. After 40 min, water is added and the reaction mixture is extracted with dichloromethane. The organic phase is washed with saturated aqueous sodium chloride solution, dried and concentrated under reduced pressure. This gives 160.4 mg of the target product (91.3% of theory).

LC-MS (method 3): Rₜ=2.66 min; m/z=277 (M−H₂O)⁺

¹H-NMR (400 MHz, DMSO-d₆): δ=7.34-7.17 (m, 7H), 6.98 (d, 2H), 4.72 (t, 1H), 4.12 (d, 2H), 3.80 (s, 3H), 2.39 (s, 3H).

### Example 13A

### Methyl 4-bromo-2-methyl-5-phenylfuran-3-carboxylate

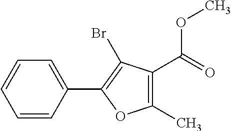

1.0 g (4.63 mmol) of methyl 2-methyl-5-phenylfuran-3-carboxylate (commercially available or obtainable by methanolysis of 2-methyl-5-phenylfuran-3-carbonyl chloride) are suspended in 5 ml of acetonitrile, and 905 mg (5.09 mmol) of N-bromosuccinimide are added with cooling at −20° C. After the addition has ended, the mixture is warmed to 0° C. and, after 30 min, to RT and then concentrated to dryness under reduced pressure. From the residue, the product is isolated by preparative RP-HPLC (acetonitrile/water). This gives 1.13 g of the target product (83.1% of theory).

LC-MS (method 3): Rₜ=2.97 min; m/z=294/296 (M+H)⁺

¹H-NMR (400 MHz, DMSO-d₆): δ=7.90 (d, 2H), 7.54-7.40 (m, 3H), 3.84 (s, 3H), 2.63 (s, 3H).

### Example 14A

### Methyl 4-(4-methoxyphenyl)-2-methyl-5-phenylfuran-3-carboxylate

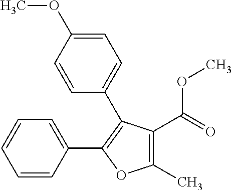

Under argon, 1260 mg (4.27 mol) of methyl 4-bromo-2-methyl-5-phenylfuran-3-carboxylate are dissolved in 5.0 ml of DMF, and 713.6 mg (4.70 mmol) of 4-methoxyphenylboronic acid, 4.3 ml (8.6 mmol) of 2N aqueous sodium carbonate solution and 150 mg (0.21 mmol) of bis(triphenylphosphine)palladium(II) chloride are added in succession. With vigorous stirring, the mixture is heated at 80° C. for 3 h. After cooling, the crude mixture is diluted with dichloromethane/methanol and filtered through kieselguhr (which is then washed with dichloromethane/methanol). The filtrate is concentrated to dryness, triturated with dichloromethane and filtered again. The filtrate is concentrated and the residue is purified by chromatography on silica gel (cyclohexane/ethyl acetate 50:1). This gives 1110 mg of the target product (80.7% of theory).

LC-MS (method 3): Rₜ=3.13 min; m/z=345 (M+Na)⁺

¹H-NMR (400 MHz, DMSO-d₆): δ=7.32-7.21 (m, 5H), 7.18 (d, 2H), 6.97 (d, 2H), 3.81 (s, 1H), 3.59 (s, 3H), 2.63 (s, 3H).

### Example 15A

### 4-(4-Methoxyphenyl)-2-methyl-5-phenylfuran-3-carboxylic acid

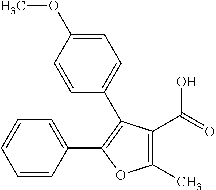

250 mg (0.776 mmol) of methyl 4-(4-methoxyphenyl)-2-methyl-5-phenylfuran-3-carboxylate are dissolved in 6 ml of THF/Methanol 1:1, and 2.5 ml of 1N aqueous sodium hydroxide solution are added at RT. The solution is stirred at RT for 1 h (hardly any conversion). After addition of 5 ml of 10% strength aqueous sodium hydroxide, the suspension is warmed to 50° C. and stirred vigorously for 1 h. After cooling, 10 ml of 1N aqueous sodium hydroxide solution are added and the aqueous phase is extracted with 30 ml of methyl tert-butyl ether. The organic phase is separated off and discarded. The aqueous phase is acidified carefully with conc. hydrochloric acid, and the resulting suspension is extracted with methyl tert-butyl ether. The organic phase is washed with saturated aqueous sodium chloride solution, dried over sodium sulfate and concentrated under reduced pressure, and the solid is concentrated under high vacuum. This gives 113.0 mg of the target product (47.3% of theory).

LC-MS (method 4): Rₜ=1.31 min; m/z=309 (M+H)⁺

¹H-NMR (400 MHz, DMSO-d₆): δ=12.33 (br s, 1H), 7.30-7.20 (m, 5H), 7.19 (d, 2H), 6.95 (d, 2H), 3.80 (s, 3H), 2.63 (s, 3H).

### Example 16A

### 6-Methoxy-6-oxohexyl 2-methyl-5-phenylfuran-3-carboxylate

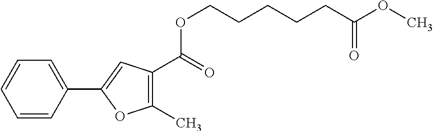

Under argon, 994 mg (6.8 mmol) of methyl 6-hydroxyhexanoate are dissolved in 5 ml of dichloromethane, 0.76 ml (5.44 mmol) of triethylamine and 55 mg (0.45 mmol) of 4-N,N-dimethylaminopyridine are added, the mixture is cooled to 0° C. and 1.0 g (4.53 mmol) of 2-methyl-5-phenylfuran-3-carbonyl chloride are added. Cooling is removed, and the reaction mixture is stirred at 0° C. for 2 h and then added to water and extracted three times with dichloromethane. The combined organic phases are washed with saturated aqueous sodium chloride solution, dried over magnesium sulfate and concentrated under reduced pressure. The crude product is purified by chromatography on silica gel (cyclohexane/ethyl acetate 10:1 to 9:1). This gives 660.2 mg of the target product (44.1% of theory)

LC-MS (method 5): Rₜ=2.71 min; m/z=331 (M+H)⁺

¹H-NMR (400 MHz, DMSO-d₆): δ=7.72 (d, 2H), 7.46-7.41 (m, 2H), 7.35-7.30 (m, 1H), 7.14 (s, 1H), 4.21 (t, 2H), 3.59 (s, 3H), 2.62 (s, 3H), 2.35 (t, 2H), 1.73-1.66 (m, 2H), 1.63-1.57 (m, 2H), 1.45-1.37 (m, 2H).

The following example can be obtained in an analogous manner starting with 2-methyl-5-phenylfuran-3-carbonyl chloride and tert-butyl(−)-6-hydroxyheptanoate:

### Example 17A

### (1R)-6-tert-Butoxy-1-methyl-6-oxohexyl 2-methyl-5-phenylfuran-3-carboxylate

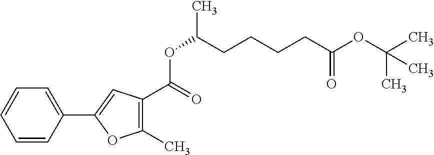

LC-MS (method 2): Rₜ=3.00 min; m/z=387 (M+H)⁺

¹H-NMR (400 MHz, DMSO-d₆): δ=7.72 (d, 2H), 7.44 (t, 2H), 7.35-7.30 (m, 1H), 7.12 (s, 1H), 5.00 (m, 1H), 2.62 (s, 3H), 2.19 (t, 2H), 1.70-1.48 (m, 4H), 1.39 (s, 9H), 1.39-1.30 (m, 2H), 1.29 (d, 3H).

[α]_{D}²⁰=−37.4°, c=0.580, chloroform.

### Example 18A

### 6-Methoxy-6-oxohexyl 4-bromo-2-methyl-5-phenylfuran-3-carboxylate

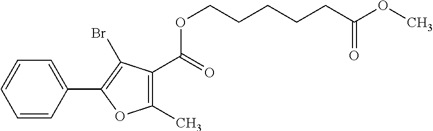

250.0 mg (0.76 mmol) of 6-methoxy-6-oxohexyl 2-methyl-5-phenylfuran-3-carboxylate are suspended in 0.8 ml of acetonitrile, and 161.2 mg (0.91 mmol) of N-bromosuccinimide are added at RT. The mixture is stirred at RT for 1 h and then concentrated under reduced pressure. The product is isolated from the crude mixture by chromatography on silica gel (cyclohexane/ethyl acetate 20:1 to 15:1). This gives 236.2 mg of the target product (76.3% of theory).

LC-MS (method 2): Rₜ=2.64 min; m/z=408 (M+H)⁺.

¹H-NMR (400 MHz, DMSO-d₆): δ=7.39 (d, 2H), 7.55-7.41 (m, 3H), 4.25 (t, 2H), 3.59 (s, 3H), 2.62 (s, 3H), 2.35 (t, 2H), 1.73-1.68 (m, 2H), 1.64-1.55 (m, 2H), 1.48-1.41 (m, 2H).

The following example can be obtained in an analogous manner starting with (1R)-6-tert-butoxy-1-methyl-6-oxohexyl 2-methyl-5-phenylfuran-3-carboxylate:

### Example 19A

### (1R)-6-tert-Butoxy-1-methyl-6-oxohexyl 4-bromo-2-methyl-5-phenylfuran-3-carboxylate

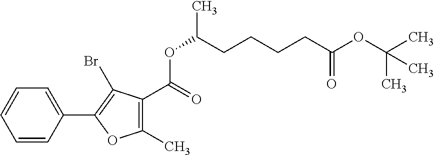

LC-MS (method 2): Rₜ=3.08 min; m/z=485/487 (M+H)⁺

¹H-NMR (400 MHz, DMSO-d₆): δ=7.92-7.88 (m, 2H), 7.58-7.42 (m, 3H), 5.05 (m, 1H), 2.62 (s, 3H), 2.19 (t, 2H), 1.70-1.50 (m, 4H), 1.41-1.37 (m, 2H), 1.39 (s, 9H), 1.39 (d, 3H).

[α]_{D}²⁰=−21.6°, c=0.575, chloroform.

### Example 20A

### Methyl 7-{[(2-methyl-5-phenylfuran-3-yl)carbonyl]amino}heptanoate

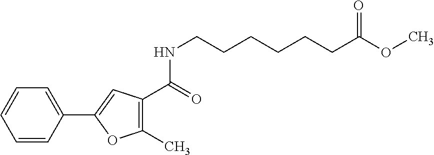

Under argon, 2.0 g (9.06 mmol) of 2-methyl-5-phenylfuran-3-carbonyl chloride are dissolved in 10 ml dichloromethane and the mixture is cooled to 0° C. At 0° C., 3.55 g (18.1 mmol) of methyl 7-aminoheptanoate hydrochloride and 3.79 ml (27.2 mmol) of triethylamine are added. The reaction mixture is slowly warmed to RT and, after 2 h, added to water and extracted three times with dichloromethane. The organic phases are combined, washed with saturated aqueous sodium chloride solution, dried over magnesium sulfate and evaporated to dryness under reduced pressure. The product is isolated from the crude mixture by chromatography on silica gel (cyclohexane/ethyl acetate 10:1 to 6:1). This gives 1.26 g of the target product (40.3% of theory).

LC-MS (method 3): Rₜ=2.70 min; m/z=344 (M+H)⁺

¹H-NMR (400 MHz, DMSO-d₆): δ=8.01 (t, 1H), 7.62 (d, 2H), 7.44 (t, 2H), 7.34-7.29 (m, 1H), 7.25 (s, 1H), 3.59 (s, 3H), 3.20 (qu, 2H), 2.59 (s, 3H), 2.31 (t, 2H), 1.59-1.48 (m, 4H), 1.35-1.28 (m, 4H).

The following example can be obtained in an analogous manner starting with 2-methyl-5-phenylfuran-3-carbonyl chloride and methyl 6-aminohexanoate hydrochloride:

### Example 21A

### Methyl 6-{[(2-methyl-5-phenylfuran-3-yl)carbonyl]amino}hexanoate

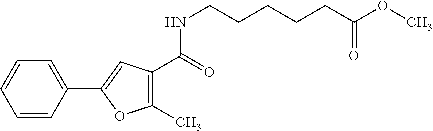

LC-MS (method 5): Rₜ=2.18 min; m/z=330 (M+H)⁺

¹H-NMR (400 MHz, DMSO-d₆): δ=8.01 (t, 1H), 7.61 (d, 2H), 7.45 (t, 2H), 7.35-7.29 (m, 1H), 7.24 (s, 1H), 3.58 (s, 3H), 2.59 (s, 3H), 2.31 (t, 3H), 1.61-1.45 (m, 4H), 1.35-1.28 (m, 2H).

### Example 22A

### Methyl 7-{[(4-bromo-2-methyl-5-phenylfuran-3-yl)carbonyl]amino}heptanoate

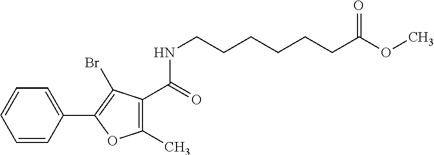

1543 mg (4.49 mmol) of methyl 7-{[(2-methyl-5-phenylfuran-3-yl)carbonyl]amino}heptanoate are suspended in 4.5 ml of 1,2-dichloroethane, the mixture is cooled to 0° C. and 960 mg (5.39 mmol) of N-bromosuccinimide are added. After 40 min, the reaction mixture is concentrated under reduced pressure and the residue is separated by chromatography on silica gel (cyclohexane/ethyl acetate 10:1 to 6:1). This gives 594.0 mg of the target product (31.3% of theory).

LC-MS (method 5): Rₜ=2.41 min; m/z=422/424 (M+H)⁺

¹H-NMR (400 MHz, DMSO-d₆): δ=8.13 (t, 1H), 7.88 (d, 2H), 7.49 (t, 2H), 7.44-7.38 (m, 1H), 3.59 (s, 3H), 3.22 (q, 2H), 2.55 (s, 3H), 2.30 (t, 2H), 1.59-1.46 (m, 4H), 1.39-1.27 (m, 4H).

### Example 23A

### Methyl 6-{[(4-bromo-2-methyl-5-phenylfuran-3-yl)carbonyl]amino}hexanoate

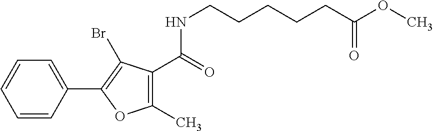

642.0 mg (1.95 mmol) of methyl 6-{[(2-methyl-5-phenylfuran-3-yl)carbonyl]amino}hexanoate are suspended in 8 ml of acetonitrile, and three portions of in total 416.3 mg (2.34 mmol) of N-bromosuccinimide are added at RT. After 35 min, the mixture is concentrated under reduced pressure and the residue is separated by chromatography on silica gel (cyclohexane/ethyl acetate 8:1 to 2:1). This gives 579.0 mg of the target product (72.8% of theory).

LC-MS (method 3): Rₜ=2.68 min; m/z=408/410 (M+H)⁺

¹H-NMR (400 MHz, DMSO-d₆): δ=8.14 (t, 1H), 7.87 (d, 2H), 7.50 (t, 2H), 7.43-7.39 (m, 1H), 3.59 (s, 3H), 3.21 (qu, 2H), 2.55 (s, 3H), 2.32 (t, 2H), 1.61-1.45 (m, 4H), 1.49-1.31 (m, 2H),

### Example 24A

### tert-Butyl(+/−)-6-{[(2-methyl-5-phenylfuran-3-yl)carbonyl]amino}heptanoate

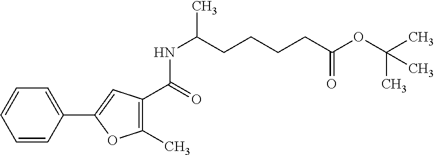

Under argon, 219.2 g (0.99 mmol) of 2-methyl-5-phenylfuran-3-carbonyl chloride are dissolved in 1.0 ml of dichloromethane and cooled to 0° C. At 0° C., 200 mg (0.99 mmol) of (+/−)-tert-butyl aminoheptanoate and 0.21 ml (1.49 mmol) of triethylamine are added. The reaction mixture is slowly warmed to RT and, after 2 h, added to water and extracted three times with dichloromethane. The organic phases are combined, washed with saturated aqueous sodium chloride solution, dried over magnesium sulfate and evaporated to dryness under reduced pressure. The product is isolated from the crude mixture by preparative RP-HPLC (acetonitrile/water). This gives 194.6 mg of the target product (50.8% of theory).

LC-MS (method 6): Rₜ=2.75 min; m/z=386 (M+H)⁺

¹H-NMR (400 MHz, DMSO-d₆): δ=7.71 (d, 1H), 7.61 (d, 2H), 7.45 (t, 2H), 7.33-7.29 (m, 2H), 3.95 (m, 1H), 2.59 (s, 3H), 2.19 (t, 2H), 1.54-1.42 (m, 4H), 1.38 (s, 9H), 1.35-1.24 (m, 2H), 1.12 (d, 3H).

The following example can be obtained in an analogous manner starting with 2-methyl-5-phenylfuran-3-carbonyl chloride and tert-butyl (6R)-6-aminoheptanoate:

### Example 25A

### tert-Butyl(−)-(6R)-6-{[(2-methyl-5-phenylfuran-3-yl)carbonyl]amino}heptanoate

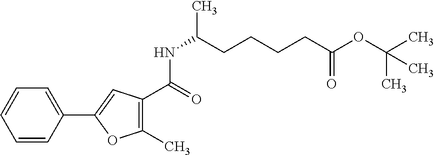

LC-MS (method 4): Rₜ=1.49 min; m/z=386 (M+H)⁺¹H-NMR (400 MHz, DMSO-d₆): δ=7.71 (d, 1H), 7.62 (d, 2H), 7.45 (t, 2H), 7.33-7.28 (m, 2H), 3.95 (m, 1H), 2.59 (s, 3H), 2.19 (t, 2H), 1.54-1.42 (m, 4H), 1.38 (s, 9H), 1.36-1.24 (m, 2H), 1.12 (d, 3H).

[α]_{D}²⁰=−23.1°, c=0.485, chloroform.

### Example 26A

### tert-Butyl(+/−)-6-{[(4-bromo-2-methyl-5-phenylfuran-3-yl)carbonyl]amino}heptanoate

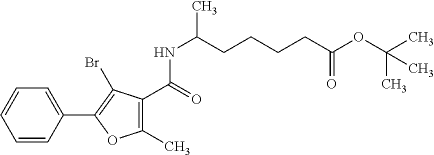

190.0 mg (0.493 mmol) of tert-butyl(+/−)-6-{[(2-methyl-5-phenylfuran-3-yl)carbonyl]-amino}heptanoate are suspended in 2 ml of acetonitrile, and three portions of in total 105.3 mg (0.591 mmol) of N-bromosuccinimide are added at RT. After 25 min of stirring, the mixture is diluted with dichloromethane and washed with saturated aqueous sodium bicarbonate solution and saturated aqueous sodium chloride solution, dried over sodium sulfate and concentrated under reduced pressure. The residue is separated by preparative RP-HPLC (acetonitrile/water). This gives 124.5 mg of the target product (50.4% of theory).

LC-MS (method 6): Rₜ=2.87 min; m/z=464/466 (M+H)⁺

¹H-NMR (400 MHz, DMSO-d₆): δ=7.99 (d, 1H), 7.87 (d, 2H), 7.51 (t, 2H), 7.44-7.39 (m, 1H), 3.93 (m, 1H), 2.54 (s, 3H), 2.20 (t, 2H), 1.55-1.30 (m, 6H), 1.20 (s, 9H), 1.12 (d, 3H).

The following example can be obtained in an analogous manner starting with tert-butyl (−)-(6R)-6-{[(2-methyl-5-phenylfuran-3-yl)carbonyl]amino}heptanoate:

### Example 27A

### tert-Butyl(−)-(6R)-6-{[(4-bromo-2-methyl-5-phenylfuran-3-yl)carbonyl]amino}heptanoate

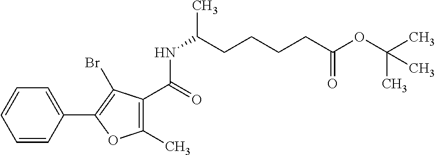

LC-MS (method 7): Rₜ=2.85 min; m/z=464/466 (M+H)⁺

¹H-NMR (400 MHz, DMSO-d₆): δ=7.99 (d, 1H), 7.87 (d, 2H), 7.50 (t, 2H), 7.44-7.39 (m, 1H), 3.92 (m, 1H), 2.54 (s, 3H), 2.20 (t, 2H), 1.55-1.30 (m, 6H), 1.20 (s, 9H), 1.12 (d, 3H).

[α]_{D}²⁰=−14.4°, c=0.515, chloroform.

### Exemplary Embodiments

### Example 1

### 6-Methoxy-6-oxohexyl 4-(4-methoxyphenyl)-2-methyl-5-phenylfuran-3-carboxylate

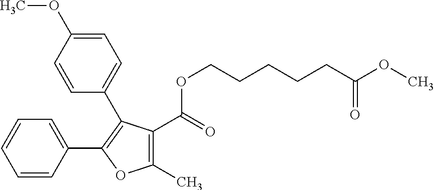

Under argon, 150 mg (0.366 mol) of 6-methoxy-6-oxohexyl 4-bromo-2-methyl-5-phenylfuran-3-carboxylate are dissolved in 0.8 ml of DMSO, and 66.8 mg (0.440 mmol) of 4-methoxyphenylboronic acid, 76.0 mg (0.55 mmol) of potassium carbonate, 80 μl of methanol and 14.4 mg (0.022 mmol) of bis(triphenylphosphine)palladium(II) chloride are added in succession. With vigorous stirring, the mixture is heated at 80° C. for about 4.5 h (reaction monitored by LC-MS). After cooling, the crude mixture is separated directly by preparative RP-HPLC and the product is isolated. This gives 50.2 mg of the target product (31.4% of theory).

LC-MS (method 3): Rₜ=3.37 min; m/z=437 (M+H)⁺

¹H-NMR (400 MHz, DMSO-d₆): δ=7.30-7.20 (m, 5H), 7.20 (d, 2H), 6.98 (d, 2H), 3.96 (t, 2H), 3.81 (s, 3H), 3.58 (s, 3H), 2.65 (s, 3H), 2.23 (t, 2H), 1.43-1.32 (m, 4H), 1.04-0.95 (m, 2H)

In an analogous manner, it is possible to prepare the following examples:

### Example 2

### Methyl 7-({[4-(4-methoxyphenyl)-2-methyl-5-phenylfuran-3-yl]carbonyl}amino)heptanoate

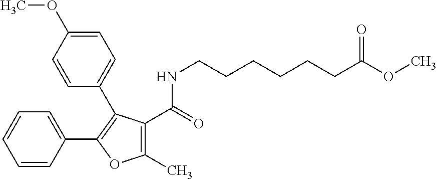

Starting with methyl 7-{[(4-bromo-2-methyl-5-phenylfuran-3-yl)carbonyl]amino}heptanoate and 4-methoxyphenylboronic acid, 84 mg of the target product (51.6% of theory) are obtained.

LC-MS (method 2): Rₜ=2.43 min; m/z=450 (M+H)⁺

¹H-NMR (400 MHz, DMSO-d₆): δ=7.34 (t, 1H), 7.31-7.28 (m, 5H), 7.23 (d, 2H), 6.98 (d, 2H), 3.79 (s, 3H), 3.58 (s, 3H), 3.05 (q, 2H), 2.55 (s, 3H), 2.28 (t, 2H), 1.50-1.40 (m, 2H), 1.30-1.04 (m, 6H).

### Example 3

### Methyl 7-({[4-(4-ethylphenyl)-2-methyl-5-phenylfuran-3-yl]carbonyl}amino)heptanoate

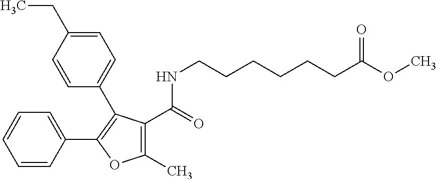

Starting with methyl 7-{[(4-bromo-2-methyl-5-phenylfuran-3-yl)carbonyl]amino}heptanoate and 4-ethylphenylboronic acid, 72.8 mg of the target product (45.8% of theory) are obtained.

LC-MS (method 2): Rₜ=2.69 min; m/z=448 (M+H)⁺

¹H-NMR (400 MHz, DMSO-d₆): δ=7.39 (t, 1H), 7.30-7.20 (m, 8H), 3.59 (s, 3H), 3.04 (qu, 2H), 2.65 (qu, 2H), 2.45 (s, 3H), 2.27 (t, 2H), 1.50-1.142 (m, 2H), 1.30-1.15 (m, 4H), 1.22 (t, 3H), 1.15-1.06 (m, 2H).

### Example 4

### Methyl 6-({[4-(4-methoxyphenyl)-2-methyl-5-phenylfuran-3-yl]carbonyl}amino)hexanoater

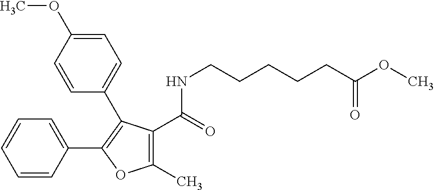

Starting with methyl 6-{[(4-bromo-2-methyl-5-phenylfuran-3-yl)carbonyl]amino}hexanoate and 4-methoxyphenylboronic acid, 43.9 mg of the target product (45.7% of theory) are obtained.

LC-MS (method 5): Rₜ=2.51 min; m/z=436 (M+H)⁺

¹H-NMR (400 MHz, DMSO-d₆): δ=7.38 (t, 1H), 7.32-7.28 (m, 5H), 7.23 (d, 2H), 6.98 (d, 2H), 3.80 (s, 3H), 3.59 (s, 3H), 3.04 (qu, 2H), 2.55 (s, 3H), 2.24 (t, 2H), 1.49-1.40 (m, 2H), 1.32-1.22 (m, 2H), 1.11-1.03 (m, 2H).

### Example 5

### Methyl 6-({[4-(4-ethylphenyl)-2-methyl-5-phenylfuran-3-yl]carbonyl}amino)hexanoate

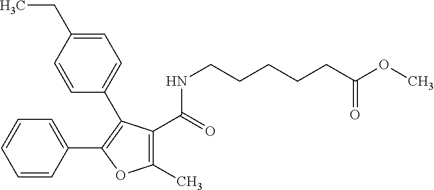

Starting with methyl 6-{[(4-bromo-2-methyl-5-phenylfuran-3-yl)carbonyl]amino}hexanoate and 4-ethylphenylboronic acid, 36.7 mg of the target product (35.3% of theory) are obtained.

LC-MS (method 2): Rₜ=2.60 min; m/z=434 (M+H)⁺

¹H-NMR (400 MHz, DMSO-d₆): δ=7.41 (t, 1H), 7.32-7.20 (m, 9H), 3.59 (s, 3H), 3.04 (qu, 2H), 2.67 (qu, 2H), 2.44 (s, 3H), 2.24 (t, 2H), 1.49-1.41 (m, 2H), 1.30-1.23 (m, 2H), 1.22 (t, 3H), 1.15-1.08 (m, 2H).

### Example 6

### (1R)-6-tert-Butoxy-1-methyl-6-oxohexyl 4-(4-methoxyphenyl)-2-methyl-5-phenylfuran-3-carboxylate

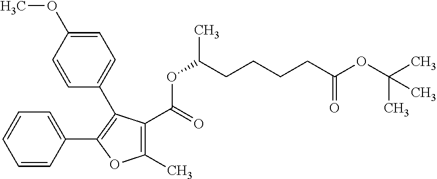

Under argon, 120 mg (0.258 mol) of (1R)-6-tert-butoxy-1-methyl-6-oxohexyl 4-bromo-2-methyl-5-phenylfuran-3-carboxylate are dissolved in 300 μl of DMSO, and 47.0 mg (0.309 mmol) of 4-methoxyphenylboronic acid, 260 μl 2N of aqueous sodium carbonate solution and 10.9 mg (0.015 mmol) of bis(triphenylphosphine)palladium(II) chloride are added in succession. With vigorous stirring, the mixture is heated at 80° C. for about 1.5 h (reaction monitored by LC-MS). After cooling, the reaction mixture is separated directly by preparative RP-HPLC and the product is isolated. This gives 71.4 mg of the target product (80% pure, 45.0% of theory)

LC-MS (method 2): R=3.15 min; m/z=493 (M+H)⁺

¹H-NMR (400 MHz, DMSO-d₆): δ=7.30-7.20 (m, 5H), 7.18 (d, 2H), 6.98 (d, 2H), 4.78 (m, 1H), 3.80 (s, 3H), 2.64 (s, 3H), 2.11 (t, 2H), 1.71-1.57 (m, 2H), 1.38 (s, 9H), 1.40-1.25 (m, about 2H), 1.06-0.97 (m, about 2H), 1.02 (d, 3H).

The following example can be obtained in an analogous manner starting with (1R)-6-tert-butoxy-1-methyl-6-oxohexyl 4-bromo-2-methyl-5-phenylfuran-3-carboxylate and 4-ethylphenylboronic acid:

### Example 7

### (1R)-6-tert-Butoxy-1-methyl-6-oxohexyl 4-(4-ethylphenyl)-2-methyl-5-phenylfuran-3-carboxylate

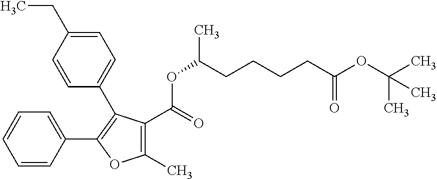

47.3 mg of the target product (29.9% of theory) are obtained.

LC-MS (method 5): Rₜ=3.52 min; m/z=513 (M+Na)⁺

¹H-NMR (400 MHz, DMSO-d₆): δ=7.28-7.21 (m, 6H), 7.18 (d, 2H), 4.77 (m, 1H), 2.67 (qu, 2H), 2.62 (s, 3H), 2.11 (t, 2H), 1.38 (s, 9H), 1.38-1.30 (m, 3H), 1.23 (t, 3H), 1.20-1.11 (m, 2H), 1.06-0.96 (m, 2H), 0.98 (d, 3H).

[α]_{D}²⁰=−33.7°, c=0.485, chloroform.

### Example 8

### Ethyl 6-{[4-(4-methoxyphenyl)-2-methyl-5-phenylfuran-3-yl]methoxy}hexanoate

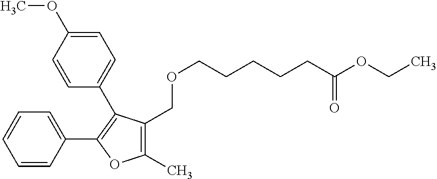

160 mg (0.544 mmol) of [4-(4-methoxyphenyl)-2-methyl-5-phenylfuran-3-yl]methanol and 157.6 mg (0.707 mmol) of ethyl 6-bromohexanoate are dissolved in 1.0 ml of absolute DMF, the mixture is cooled to 0° C. and 0.6 ml (0.6 mmol) of phosphazene base P4-t-Bu (1 M solution in hexane) are added dropwise. After 1 h at 0° C., water is added to the mixture and the solution is adjusted to approximately neutral using a 1N hydrochloric acid solution (pH about 7). The mixture is extracted with dichloromethane, and the organic phase is dried with sodium sulfate and concentrated under reduced pressure. The product is isolated by preparative RP-HPLC (acetonitrile/water). This gives 44.3 mg of the target product (18.7% of theory).

MS (DCI); m/z=454 (M+NH₄)⁺

¹H-NMR (400 MHz, DMSO-d₆): δ=7.33-7.19 (m, 7H), 6.98 (d, 2H), 4.09 (s, 2H), 4.03 (qu, 2H), 3.80 (s, 3H), 3.30 (qu, 2H), 2.39 (s, 3H), 2.26 (t, 2H), 1.55-1.42 (m, 4H), 1.29-1.20 (m, 2H), 1.15 (t, 3H).

### Example 9

### Methyl[3-({[4-(4-methoxyphenyl)-2-methyl-5-phenylfuran-3-yl]carbonyl}amino)phenoxy]acetate

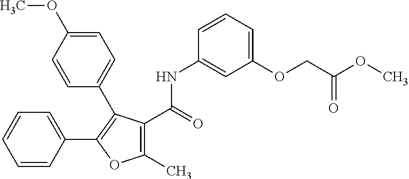

110 mg (0.357 mmol) of 4-(4-methoxyphenyl)-2-methyl-5-phenylfuran-3-carboxylic acid are dissolved in 0.5 ml of DMF, and 149.2 mg (0.392 mmol) of HATU are added. The mixture is stirred at RT for 10 min, and 80.8 mg (0.446 mmol) of methyl 3-aminophenoxyacetate, 4.4 mg (0.036 mmol) of 4-N,N-dimethylaminopyridine and 75 μl (0.428 mmol) of diisopropylethylamine are then added. The reaction mixture is stirred at RT overnight and then added to water. The aqueous phase is extracted three times with ethyl acetate, and the combined organic phases are dried over magnesium sulfate and concentrated under reduced pressure. The product is isolated from the crude mixture by preparative RP-HPLC (acetonitrile/water). This gives 120 mg of the target product (slightly contaminated, about 70% of theory).

LC-MS (method 2): Rₜ=2.49 min; m/z=472 (M+H)⁺

¹H-NMR (400 MHz, DMSO-d₆): δ=9.85 (s, 1H), 7.38-7.05 (m, 10H), 6.98 (d, 2H), 6.61 (d, 1H), 4.73 (s, 2H), 3.78 (s, 3H), 3.32 (s, 3H), 2.58 (s, 3H).

### Example 10

### Methyl(+/−)-6-({[4-(4-methoxyphenyl)-2-methyl-5-phenylfuran-3-yl]methyl}amino)heptanoate

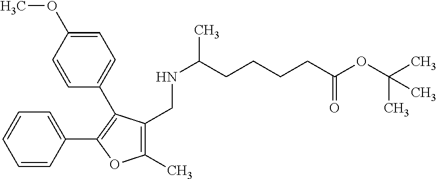

150 mg (513 mmol) of 4-(4-methoxyphenyl)-2-methyl-5-phenylfuran-3-carbaldehyde are dissolved in 0.5 ml of absolute THF, and 113.6 mg (0.564 mmol) of tert-butyl(+/−)-aminoheptanoate and 0.30 ml (1.026 mmol) of titanium(IV) isopropoxide are added. The mixture is stirred at RT overnight and then cooled to 10° C., and 38.8 mg (1.026 mmol) of sodium borohydride are added. After about 1 h, 0.58 ml of ethanol is slowly added dropwise with cooling. The mixture is warmed to RT and stirred for 2 h, and a little water is then added. The resulting white precipitate is filtered off with suction and washed three times with water and in each case twice with methanol and dichloromethane. The filtrate is diluted with water, and the organic phase is separated off. The aqueous phase is extracted twice with dichloromethane. All organic phases are combined, dried over magnesium sulfate and concentrated under reduced pressure. The product is purified by preparative RP-HPLC (acetonitrile/water). This gives 26.6 mg of the target product (10.9% of theory).

LC-MS (method 3): Rₜ=2.17 min; m/z=478 (M+H)⁺

¹H-NMR (400 MHz, DMSO-d₆): δ=7.34-7.15 (m, 7H), 6.99 (d, 2H), 3.81 (s, 3H), 2.45-2.32 (m, about 2H), 2.35 (s, 3H), 2.15 (t, 2H), 1.45-1.35 (m, 2H), 1.39 (s, 9H), 1.28-1.12 (m, 4H), 0.87 (d, 3H).

### Example 11

### tert-Butyl(+/−)-6-({[4-(4-methoxyphenyl)-2-methyl-5-phenylfuran-3-yl]carbonyl}amino)heptanoate

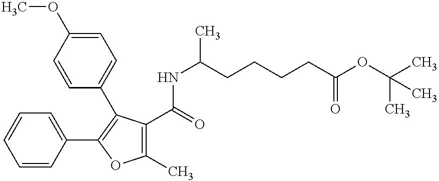

Under argon, 122 mg (0.263 mol) of tert-butyl(+/−)-6-{[(4-bromo-2-methyl-5-phenylfuran-3-yl)carbonyl]amino}heptanoate are dissolved in 615 μl of DMSO, and 47.9 mg (0.315 mmol) of 4-methoxyphenylboronic acid, 54.5 mg (0.55 mmol) of potassium carbonate, 62 μl of methanol and 11.1 mg (0.016 mmol) of bis(triphenylphosphine)palladium(II) chloride are added in succession. With vigorous stirring, the mixture is heated at 80° C. for 2 h. A further 24 mg (0.158 mmol) of 4-methoxyphenylboronic acid are added, and the reaction mixture is stirred at 80° C. for a further 1.5 h. After cooling, the reaction mixture is separated directly by preparative RP-HPLC and the product is isolated. This gives 55.1 mg of the target product (42.7% of theory).

LC-MS (method 2): Rₜ=2.80 min; m/z=492 (M+H)⁺

¹H-NMR (400 MHz, DMSO-d₆): δ=7.32-7.20 (m, 7H), 7.12 (d, 1H), 6.99 (d, 2H), 3.81 (s, 3H), 3.81-3.72 (m, 1H), 2.46 (s, 3H), 2.11 (t, 3H), 1.42-1.36 (m, 2H), 1.38 (s, 9H), 1.29-1.19 (m, 2H), 1.11-1.03 (m, 2H), 0.94 (d, 3H).

Separation of the enantiomers: The racemic mixture (40 mg) of tert-butyl(+/−)-6-({[4-(4-methoxyphenyl)-2-methyl-5-phenylfuran-3-yl]carbonyl}amino)heptanoate is dissolved in a mixture of 2 ml of isohexane and 0.2 ml of ethanol and separated into the enantiomers by chromatography on a chiral phase; column: Daicel Chiralpak AD-H, 5 μm, 250 mm×30 mm; flow rate: 15 ml/min; detection: 220 nm; injection volume: 1.1 ml; temperature: 30° C.; mobile phase: 95% isohexane/5% ethanol. The following examples are isolated:

### Example 12

### tert-Butyl(+)-(6S)-({[4-(4-methoxyphenyl)-2-methyl-5-phenylfuran-3-yl]carbonyl}amino)heptanoate

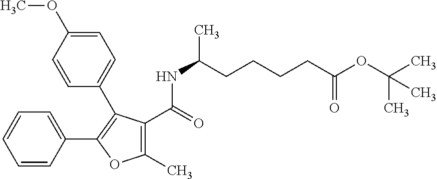

7.0 mg of the target product (35% of theory) are obtained.

¹H-NMR (400 MHz, CDCl₃): δ=7.35-7.15 (m, 7H), 7.04 (d, 2H), 5.04 (d, 1H), 3.95-3.88 (m. about 1H) 3.90 (s, 3H), 2.72 (s, 3H), 2.16 (t, 3H), 1.52-1.40 (m, about 2H), 1.46 (s, 9H), 1.30-1.02 (m, about 4H), 0.89 (d, 3H).

[α]_{D}²⁰=+42.5°, c=0.35, chloroform.

and

### Example 13

### tert-Butyl(−)-(6R)-6-({[4-(4-methoxyphenyl)-2-methyl-5-phenylfuran-3-yl]carbonyl}amino)heptanoate

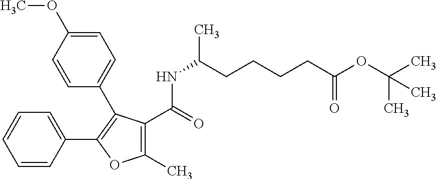

9.0 mg of the target product (45% of theory) are obtained.

¹H-NMR (400 MHz, CDCl₃): δ=7.35-7.15 (m, 7H), 7.03 (d, 2H), 5.03 (d, 1H), 3.95-3.88 (m, 1H) 3.90 (s, 3H), 2.72 (s, 3H), 2.16 (t, 3H), 1.52-1.40 (m, about 2H), 1.46 (s, 9H), 1.30-1.02 (m, 4H), 0.88 (d, 3H).

[α]_{D}²⁰=−31.8°, c=0.450, chloroform.

Alternatively, tert-butyl(−)-(6R)-6-({[4-(4-methoxyphenyl)-2-methyl-5-phenylfuran-3-yl]carbonyl}amino)heptanoate can also be prepared by the following procedure:

Under argon, 80.0 mg (0.172 mmol) of tert-butyl(−)-(6R)-6-{[(4-bromo-2-methyl-5-phenylfuran-3-yl)carbonyl]amino}heptanoate are dissolved in 450 μl of DMSO, and 31.4 mg (0.207 mmol) of 4-methoxyphenylboronic acid, 172 μl of 2N aqueous sodium carbonate solution and 6.0 mg (0.009 mmol) of bis(triphenylphosphine)palladium(II) chloride are added in succession. With vigorous stirring, the mixture is heated at 100° C. for 2.5 h. After cooling, the product is isolated directly from the reaction mixture by preparative RP-HPLC. This gives 70.5 mg of the target product (83.3% of theory).

[α]_{D}²⁰=−28.3°, c=0.450, chloroform.

### General Procedure A: Hydrolysis of Methyl or Ethyl Esters to the Corresponding Carboxylic Acid Derivatives

At RT, 1.5 to 10 eq. of sodium hydroxide, as a 1 N aqueous solution, are added to a solution of the methyl or ethyl ester in THF or THF/methanol (1:1) (concentration about 0.05 to 0.5 mol/l). The mixture is stirred at RT for a period of 0.5-18 h and then neutralized or acidified slightly with 1 N hydrochloric acid. If a solid precipitates out, the product can be isolated by filtration, washing with water and drying under high vacuum. Alternatively, the target compound is isolated directly from the crude product, if appropriate after extractive work-up with dichloromethane, by preparative RP-HPLC (mobile phase: water/acetonitrile gradient) or by stirring with an inert solvent.

The following examples are prepared in accordance with the General Procedure A:

| Example | Structure | Analytical Data |
|---|---|---|
| 14 | 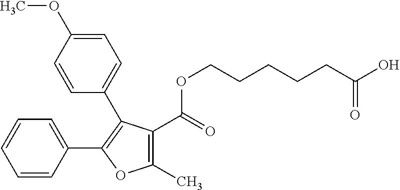 | LC-MS (method 5): Rₜ = 2.57 min; m/z = 423 (M + H)⁺ ¹H-NMR (400 MHz, DMSO-d₆): δ = 7.28- 7.20 (m, 5 H), 7.18 (d, 2 H), 6.96 (d, 2 H), 3.96 (t, 2 H), 3.81 (s, 3 H), 3.30 (qu, 2 H), 2.62 (s, 3 H), 1.81 (t, 2 H), 1.30-1.30 (m, 4 H), 1.08-1.00 (m, 2 H). |
| 15 | 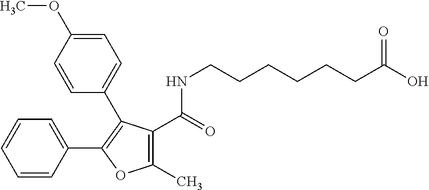 | LC-MS (method 5): Rₜ = 2.27 min; m/z = 436 (M + H)⁺ ¹H-NMR (400 MHz, DMSO-d₆): δ = 11.97 (br s, 1 H), 7.34 (t, 1 H), 7.30-7.24 (m, 5 H), 7.23 (d, 2 H), 6.98 (d, 2 H), 3.80 (s, 3 H), 3.05 (q, 2 H), 2.55 (s, 3 H), 2.21 (t, 2 H), 1.49-1.40 (m, 2 H), 1.30-1.13 (m 4 H), 1.12-1.04 (m, 2 H). |
| 16 | 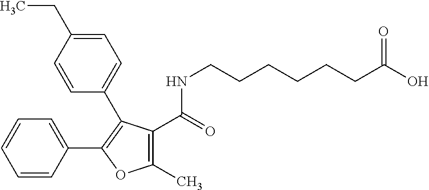 | LC-MS (method 2): Rₜ = 2.34 min; m/z = 434 (M + H)⁺ ¹H-NMR (400 MHz, DMSO-d₆): δ = 11.98 (br s, 1 H), 7.39 (t, 1 H), 7.33-7.20 (m, 9 H), 3.04 (qu, 2 H), 2.67 (qu, 2 H), 2.45 (s, 3 H), 2.28 (t, 2 H), 1.49-1.141 (m, 2 H), 1.30-1.05 (m, 9 H). |
| 17 | 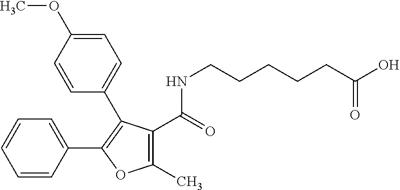 | LC-MS (method 2): Rₜ = 2.01 min; m/z = 422 (M + H)⁺ ¹H-NMR (400 MHz, CDCl₃): δ = 7.28- 7.10 (m, about 7 H), 6.98 (d, 2 H), 5.40 (t, 1 H), 3.80 (s, 3 H), 3.04 (qu, 2 H), 2.59 (s, 3 H), 2.01 (t, 2 H), 1.45-1.35 (m, 2 H), 1.17-1.08 (m, 2 H), 1.04-0.95 (m, 2 H). |
| 18 | 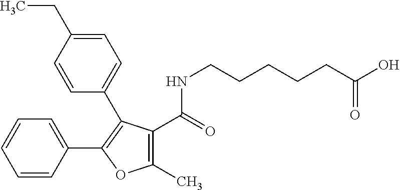 | LC-MS (method 5): Rₜ = 2.41 min; m/z = 420 (M + H)⁺ ¹H-NMR (400 MHz, DMSO-d₆): δ = 11.99 (s, 1 H), 7.41 (t, 1 H), 7.31-7.20 (m, 9 H), 3.05 (qu, 2 H), 2.65 (qu, 2 H), 2.45 (s, 3 H), 2.15 (t, 2 H), 1.45-1.39 (m, 2 H), 1.30-1.20 (m, 2 H), 1.24 (t, 3 H), 1.15- 1.08 (m, 2 H). |
| 19 | 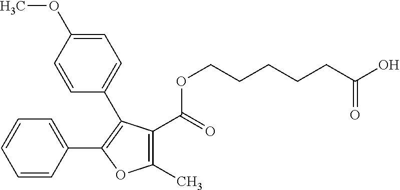 | LC-MS (method 2): Rₜ = 2.42 min; m/z = 431 (M + Na)⁺ ¹H-NMR (500 MHz, CDCl₃): δ = 7.40 (d, 2 H), 7.31-7.12 (m, 6 H), 6.92 (d, 2 H), 4.16 (s, 2 H), 3.85 (s, 3 H), 3.36 (t, 2 H), 2.42 (s, 3 H), 2.34 (t, 2 H), 1.65-1.52 (m, H), 1.40-1.32 (m, 2 H), |
| 20 | 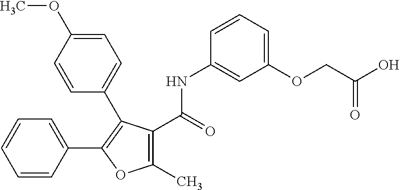 | LC-MS (method 3): Rₜ = 2.72 min; m/z = 458 (M + H)⁺ ¹H-NMR (400 MHz, DMSO-d₆): δ = 13.02 (br s, 1 H), 9.83 (s, 1 H), 7.37-7.08 (m, 10 H), 6.98 (d, 2 H), 6.60 (dd, 1 H), 4.61 (s, 2 H), 3.29 (s, 3 H), 2.58 (s, about 3 H). |

### General Procedure B: Hydrolysis of Tert-Butyl Esters to the Corresponding Carboxylic Acid Derivatives

At 0 C to RT, TFA is added dropwise to a solution of the tert-butyl ester in dichloromethane (concentration 0.1 to 1.0 mol/l, additionally optionally a drop of water) until a dichloromethane/TFA ratio of about 2:1 to about 1:2 is reached. The mixture is stirred at RT for 1-18 h and then concentrated under high vacuum. Alternatively, the mixture is diluted with dichloromethane, washed with water and saturated aqueous sodium chloride solution, dried and concentrated under reduced pressure. If required, the reaction product can be purified, for example by preparative RP-HPLC (mobile phase: acetonitrile/water gradient).

The following examples are prepared in accordance with the General Procedure B:

| Example | Structure | Analytical Data |
|---|---|---|
| 21 | 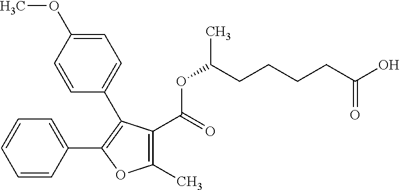 | LC-MS (method 3): Rₜ = 3.10 min; m/z = 437 (M + H)⁺ ¹H-NMR (400 MHz, DMSO-d₆): δ = 12.1 (br s, about 1 H), 7.30-7.20 (m, 5 H), 7.18 (d, 2 H), 6.97 (d, 2 H), 4.78 (m, 1 H), 3.80 (s, 3 H), 2.65 (s, 3 H), 2.11 (t, 2 H), 1.40-1.15 (m, 4 H), 1.08-0.98 (m, 2 H), 1.02 (d, 3 H). [a]_{D}²⁰ = −28.6°, c = 0.595, chloroform. |
| 22 | 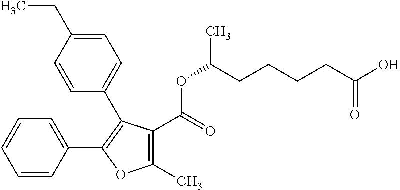 | LC-MS (method 5): Rₜ = 2.91 min; m/z = 435 (M + H)⁺, 457 (M + Na)⁺ ¹H-NMR (400 MHz, DMSO-d₆): δ = 11.98 (s, 1 H), 7.29-7.15 (m, 9 H), 4.76 (m, 1 H), 2.68 (qu, 2 H), 2.62 (s, 3 H), 2.24 (t, 2 H), 1.40-1.30 (m, 3 H), 1.25 (t, 3 H), 1.24-1.11 (m, 2 H), 1.05-1.00 (m, 2 H), 0.99 (d, 3 H). [a]_{D}²⁰ = −29.9°, c = 0.450, chloroform. |
| 23 | 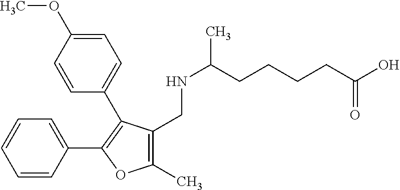 | LC-MS (method 2): Rₜ = 1.36 min; m/z = 422 (M + H)⁺ ¹H-NMR (400 MHz, CDCl₃): δ = 7.35 (m, 2 H), 7.28-7.15 (m, 6 H), 7.01 (d, 2 H), 4.04-3.97 (m, 1 H) 3.92-3.88 (m, 1 H), 3.88 (s, 3 H), 2.43 (s, 3 H), 2.35-2.20 (m, 2 H), 1.40-1.10 (m, 6 H), 1.02 (d, 3 H). |
| 24 | 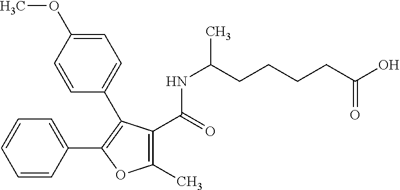 | LC-MS (method 2): Rₜ = 2.13 min; m/z = 436 (M + H)⁺ ¹H-NMR (400 MHz, CDCl₃): δ = 7.32- 7.15 (m, 8 H), 7.02 (d, 2 H), 5.04 (d, 1 H), 3.96-3.90 (m, 1 H), 3.80 (s, 3 H), 2.70 (s, 3 H), 2.29 (t, 3 H), 1.57-1.50 (m, 2 H), 1.20-1.04 (m, 4 H), 0.88 (d, 3 H). |
| 25 | 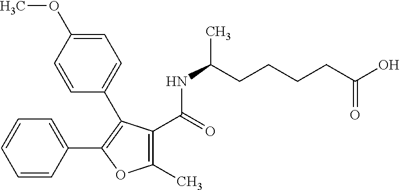 | LC-MS (method 4): Rₜ = 1.31 min; m/z = 436 (M + H)⁺. |
| 26 | 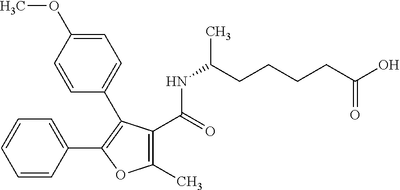 | LC-MS (method 4): Rₜ = 1.32 min; m/z = 436 (M + H)⁺ 1H-NMR (400 MHz, DMSO-d6): δ = 7.34- 7.21 (m, 7 H), 7.18 (d, 1 H), 6.98 (d, 2 H), 3.81 (s, 3 H), 3.81- 3.75 (m, 1 H), 2.45 (s, 3 H), 2.15 (t, 2 H), 1.45-1.37 (m, 2 H), 1.30-1.19 (m, 2 H), 1.15-1.07 (m, 2 H), 0.93 (d, 3 H). [a]_{D}²⁰ = −23.7°, c = 0.750, chloroform. |

### B. ASSESSMENT OF PHARMACOLOGICAL EFFICACY

The pharmacological action of the compounds according to the invention can be demonstrated in the following assays:

### B-1. Studies of Binding to Prostacyclin Receptors (IP Receptors) of Human Thrombocyte Membranes

Thrombocyte membranes are obtained by centrifuging 50 ml human blood (Buffy coats with CDP Stabilizer, from Maco Pharma, Langen) for 20 min at 160×g. Remove the supernatant (platelet-rich plasma, PRP) and then centrifuge again at 2000×g for 10 min at room temperature. Resuspend the sediment in 50 mM tris(hydroxymethyl)aminomethane, which has been adjusted to a pH of 7.4 with 1 N hydrochloric acid, and store at −20° C. overnight. On the next day, centrifuge the suspension at 80 000×g and 4° C. for 30 min. Discard the supernatant. Resuspend the sediment in 50 mM tris(hydroxymethyl)aminomethane/hydrochloric acid, 0.25 mM ethylene diamine tetraacetic acid (EDTA), pH 7.4, and then centrifuge once again at 80 000×g and 4° C. for 30 min. Take up the membrane sediment in binding buffer (50 mM tris(hydroxymethyl)-aminomethane/hydrochloric acid, 5 mM magnesium chloride, pH 7.4) and store at −70° C. until the binding test.

For the binding test, incubate 3 nM ³H-Iloprost (592 GBq/mmol, from AmershamBioscience) for 60 min with 300-1000 μg/ml human thrombocyte membranes per charge (max. 0.2 ml) in the presence of the test substances at room temperature. After stopping, add cold binding buffer to the membranes and wash with 0.1% bovine serum albumin. After adding Ultima Gold Scintillator, quantify the radioactivity bound to the membranes using a scintillation counter. The nonspecific binding is defined as radioactivity in the presence of 1 μM Iloprost (from Cayman Chemical, Ann Arbor) and is as a rule <25% of the bound total radioactivity. The binding data (IC₅₀ values) are determined using the program GraphPad Prism Version 3.02.

Representative results for the compounds according to the invention are shown in Table 1:

TABLE 1

| Example No. | IC₅₀ [nM] |
|---|---|
| 4 | 470 |
| 18 | 138 |
| 20 | 438 |
| 21 | 315 |
| 24 | 476 |
| 26 | 670 |

### B-2. IP-Receptor Stimulation on Whole Cells

The IP-agonistic action of test substances is determined by means of the human erythroleukaemia line (HEL), which expresses the IP-receptor endogenously [Murray, R., FEBS Letters 1989, 1: 172-174]. For this, the suspension cells (4×10⁷ cells/ml) are incubated with the particular test substance for 5 minutes at 30° C. in buffer [10 mM HEPES (4-(2-hydroxyethyl)-1-piperazineethanesulphonic acid)/PBS (phosphate-buffered saline, from Oxoid, UK)], 1 mM calcium chloride, 1 mM magnesium chloride, 1 mM IBMX (3-isobutyl-1-methylxanthine), pH 7.4. Next, the reaction is stopped by addition of 4° C. cold ethanol and the charges are stored for a further 30 minutes at 4° C. Then the samples are centrifuged at 10 000×g and 4° C. The resultant supernatant is discarded and the sediment is used for determination of the concentration of cyclic adenosine monophosphate (cAMP) in a commercially available cAMP-radioimmunoassay (from IBL, Hamburg). In this test, IP agonists lead to an increase in cAMP concentration, but IP antagonists have no effect. The effective concentration (EC₅₀ value) is determined using the program GraphPad Prism Version 3.02.

### B-3. Inhibition of Thrombocyte Aggregation In Vitro

Inhibition of thrombocyte aggregation is determined using blood from healthy test subjects of both sexes. Mix 9 parts blood with one part 3.8% sodium citrate solution as coagulant. Centrifuge the blood at 900 rev/min for 20 min. Adjust the pH value of the platelet-rich plasma obtained to pH 6.5 with ACD solution (sodium citrate/citric acid/glucose). Then remove the thrombocytes by centrifugation, take up in buffer and centrifuge again. Take up the thrombocyte deposit in buffer and additionally resuspend with 2 mmol/l calcium chloride.

For the measurements of aggregation, incubate aliquots of the thrombocyte suspension with the test substance for 10 min at 37° C. Next, aggregation is induced by adding ADP and is determined by the turbidimetric method according to Born in the aggregometer at 37° C. [Born G. V. R., J. Physiol. (London) 168, 178-179 (1963)].

### B-4. Measurement of Blood Pressure of Anesthetized Rats

Anesthetize male Wistar rats with a body weight of 300-350 g with thiopental (100 mg/kg i.p.). After tracheotomy, catheterize the arteria femoralis for blood pressure measurement. Administer the test substances as solution, orally by esophageal tube or intravenously via the femoral vein in a suitable vehicle.

### B-5. PAH Model in the Anesthetized Dog

In this animal model of pulmonary arterial hypertension (PAH), mongrel dogs having a body weight of about 25 kg are used. Narcosis is induced by slow i.v. administration of 25 mg/kg of sodium thiopental (Trapanal®) and 0.15 mg/kg of alcuronium chloride (Alloferin®) and maintained during the experiment by continuous infusion of 0.04 mg/kg/h of Fentanyl®, 0.25 mg/kg/h of droperidol (Dehydro-benzperidol®) and 15 μg/kg/h of alcuronium chloride (Alloferin®). Reflectory effects on the pulse by lowering of the blood pressure are kept to a minimum by autonomous blockage [continuous infusion of atropin (about 10 μg/kg/h) and propranolol (about 20 μg/kg/h)]. After intubation, the animals are ventilated using a ventilator with constant tidal volume such that an end-tidal CO₂ concentration of about 5% is reached. Ventilation takes place with ambient air enriched with about 30% oxygen (normoxa). For measuring the hemodynamic parameters, a liquid-filled catheter is implanted into the femoralis artery for measuring the blood pressure. A double-lumiger Swan-Ganz® catheter is introduced via the jugulara vein into the pulmonary artery (distal lumen for measuring the pulmonary arterial pressure, proximal lumen for measuring the central venus pressure). The left-ventricular pressure is measured following introduction of a micro-tip catheter (Millar® Instruments) via the carotis artery into the left ventricle, and from this, the dP/dt value is derived as a measure for the contractility. Substances are administered i.v. via the femoralis vein. The hemodynamic signals are recorded and evaluated using pressure sensors/amplifiers and PONEMAH® as data acquisition software.

To induce acute pulmonary hypertension, the stimulus used is either hypoxia or continuous infusion of thromboxan A₂ or a thromboxan A₂ analog. Acute hypoxia is induced by gradually reducing the oxygen in the ventilation air to about 14%, such that the mPAP increases to values of >25 mm Hg. If the stimulus used is a thromboxan A₂ analog, 0.21-0.32 μg/kg/min of U-46619 [9,11-dideoxy-9α,11α-epoxymethanoprostaglandin F_{2α} (from Sigma)] are infused to increase the mPAP to >25 mm Hg.

B-6. PAH Model in Anesthetized Göttingen Minipig

In this animal model of pulmonary arterial hypertension (PAH), göttingen minipigs having a body weight of about 25 kg are used. Narcosis is induced by 30 mg/kg of ketamine (Ketavet®) i.m., followed by i.v. administration of 10 mg/kg of sodium thiopental (Trapanal®); during the experiment, it is maintained by inhalation narcosis using enfluran (2-2.5%) in a mixture of ambient air enriched with about 30-35% oxygen/N₂O (1:1.5). For measuring the hemodynamic parameters, a liquid-filled catheter is implanted into the carotis artery for measuring the blood pressure. A double-lumiger Swan-Ganz® catheter is introduced via the jugulara vein into the pulmonary artery (distal lumen for measuring the pulmonary arterial pressure, proximal lumen for measuring the central venus pressure). The left-ventricular pressure is measured following introduction of a micro-tip catheter (Millar® Instruments) via the carotis artery into the left ventricle, and from this, the dP/dt value is derived as a measure for the contractility. Substances are administered i.v. via the femoralis vein. The hemodynamic signals are recorded and evaluated using pressure sensors/amplifiers and PONEMAH® as data acquisition software.

To induce acute pulmonary hypertension, the stimulus used is continuous infusion of a thromboxan A₂ analog. Here, 0.12-0.14 μg/kg/min of U-46619 [9,11-dideoxy-9α,11α-epoxymethanoprostaglandin F_{2α} (from Sigma)] are infused to increase the mPAP to >25 mm Hg.

### C. EXEMPLARY EMBODIMENTS OF PHARMACEUTICAL COMPOSITIONS

The compounds of the invention can be converted into pharmaceutical preparations in the following ways:

### Tablet:

### Composition:

100 mg of the compound of the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

### Production:

The mixture of compound of the invention, lactose and starch is granulated with a 5% strength solution (m/m) of the PVP in water. The granules are mixed with the magnesium stearate for 5 minutes after drying. This mixture is compressed with a conventional tablet press (see above for format of the tablet). A guideline compressive force for the compression is 15 kN.

Suspension which can be Administered Orally:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol, and the compound of the invention is added to the suspension. The water is added while stirring. The mixture is stirred for about 6 h until the swelling of the Rhodigel is complete.

Solution which can be Administered Orally:

### Composition:

500 mg of the compound of the invention, 2.5 g of polysorbate and 97 g of polyethylene glycol 400.20 g of oral solution correspond to a single dose of 100 mg of the compound according to the invention.

### Production:

The compound of the invention is suspended in the mixture of polyethylene glycol and polysorbate with stirring. The stirring process is continued until the compound according to the invention has completely dissolved.

i.v. Solution:

The compound of the invention is dissolved in a concentration below the saturation solubility in a physiologically tolerated solvent (e.g. isotonic saline solution, 5% glucose solution and/or 30% PEG 400 solution). The solution is sterilized by filtration and used to fill sterile and pyrogen-free injection containers.

1. A compound of the formula (I) 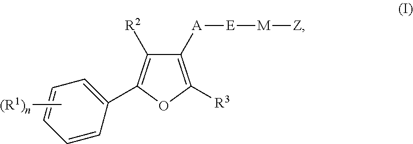
in which
A represents —CH₂— or —C(═O)—,
E represents O or NR⁴,
where
R⁴ represents hydrogen or (C₁-C₄)-alkyl,
M represents a group of the formula 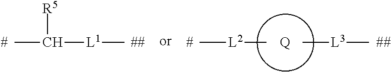
where
# represents the point of attachment to E,
## represents the point of attachment to Z,
R⁵ represents hydrogen or (C₁-C₄)-alkyl,
where alkyl may be substituted by a substituent selected from the group consisting of hydroxyl and amino,
L¹ represents (C₁-C₇)-alkanediyl, (C₂-C₇)-alkenediyl or a group of the formula *-L^{1A}-V-L^{1B}-**,
where alkanediyl and alkenediyl may be substituted by 1 or 2 fluorine substituents,
and
where
* represents the point of attachment to —CHR⁵,
** represents the point of attachment to Z,
L^{1A} represents (C₁-C₅)-alkanediyl,
where alkanediyl may be substituted by 1 or 2 substituents independently of one another selected from the group consisting of (C₁-C₄)-alkyl and (C₁-C₄)-alkoxy,
L^{1B} represents a bond or (C₁-C₃)-alkanediyl,
where alkanediyl may be substituted by 1 or 2 fluorine substituents,
and
V represents O or N—R⁶,
where
R⁶ represents hydrogen, (C₁-C₆)-alkyl or (C₃-C₇)-cycloalkyl,
L² represents a bond or (C₁-C₄)-alkanediyl,
Q represents (C₃-C₇)-cycloalkyl, (C₄-C₇)-cycloalkenyl, 5- to 7-membered heterocyclyl, phenyl or 5- or 6-membered heteroaryl,
where cycloalkyl, cycloalkenyl, heterocyclyl, phenyl and heteroaryl may be substituted by 1 or 2 substituents independently of one another selected from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino,
where alkyl may be substituted by a substituent selected from the group consisting of hydroxyl, (C₁-C₄)-alkoxy, amino, mono-(C₁-C₄)-alkylamino and di-(C₁-C₄)-alkylamino,
and
L³ represents (C₁-C₄)-alkanediyl or (C₂-C₄)-alkenediyl,
where alkanediyl may be substituted by 1 or 2 fluorine substituents,
and
wherein a methylene group of the alkanediyl group may be replaced by O or N—R⁷,
where
R⁷ represents hydrogen, (C₁-C₆)-alkyl or (C₃-C₇)-cycloalkyl,
Z represents a group of the formula 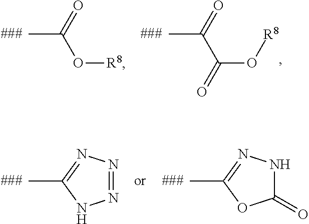
where
### represents the point of attachment to the group L¹ or L³,
and
R⁸ represents hydrogen or (C₁-C₄)-alkyl,
R¹ represents halogen, cyano, nitro, (C₁-C₆)-alkyl, trifluoromethyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethoxy, (C₁-C₆)-alkylthio, (C₁-C₆)-alkylcarbonyl, amino, mono-(C₁-C₆)-alkylamino, di-(C₁-C₆)-alkylamino or (C₁-C₆)-alkylcarbonylamino,
where (C₁-C₆)-alkyl and (C₁-C₆)-alkoxy for their part may be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
or
two radicals R¹ attached to adjacent carbon atoms of the phenyl ring together form a group of the formula —O—CH₂—O—, —O—CHF—O—, —O—CF₂—O—, —O—CH₂—CH₂—O— or —O—CF₂—CF₂—O—,
n represents the number 0, 1 or 2,
where, if R¹ is present more than once, its meaning may in each case be identical or different,
and
R² represents phenyl or 5- or 6-membered heteroaryl,
where phenyl and heteroaryl may be substituted by 1 to 3 substituents independently of one another selected from the group consisting of halogen, cyano, nitro, formyl, (C₁-C₆)-alkyl, trifluoromethyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethoxy, (C₁-C₆)-alkylthio, (C₁-C₆)-alkylcarbonyl, amino, mono-(C₁-C₆)-alkylamino, di-(C₁-C₆)-alkylamino and (C₁-C₆)-alkylcarbonylamino,
where alkyl and alkoxy may be substituted by a substituent selected from the group consisting of cyano, hydroxyl, (C₁-C₄)-alkoxy, (C₁-C₄)-alkylthio, amino, mono-(C₁-C₄)-alkylamino and di-(C₁-C₄)-alkylamino,
or
two substituents attached to adjacent carbon atoms of the phenyl ring together form a group of the formula —O—CH₂—O—, —O—CHF—O—, —O—CF₂—O—, —O—CH₂—CH₂—O— or —O—CF₂—CF₂—O—,
and
R³ represents methyl, ethyl or trifluoromethyl,
and salts thereof.

2. The compound of formula (I) as claimed in claim 1 in which
A represents —CH₂— or —C(═O)—,
E represents O or NR⁴,
where
R⁴ represents hydrogen or (C₁-C₄)-alkyl,
M represents a group of the formula 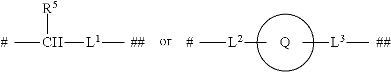
where
# represents the point of attachment to E,
## represents the point of attachment to Z,
R⁵ represents hydrogen, methyl or ethyl,
L¹ represents (C₃-C₇)-alkanediyl, (C₃-C₇)-alkenediyl or a group of the formula *-L^{1A}-V-L^{1B}-**,
where
* represents the point of attachment to —CHR⁵,
** represents the point of attachment to Z,
L^{1A} represents (C₁-C₃)-alkanediyl,
where alkanediyl may be substituted by 1 or 2 substituents independently of one another selected from the group consisting of methyl and ethyl,
L^{1B} represents (C₁-C₃)-alkanediyl,
and
V represents O or N—R⁶,
where
R⁶ represents hydrogen, (C₁-C₃)-alkyl or cyclopropyl,
L² represents a bond, methylene, ethane-1,1-diyl or ethane-1,2-diyl,
Q represents cyclobutyl, cyclopentyl, cyclopentenyl, cyclohexyl, pyrrolidinyl, piperidinyl, tetrahydrofuranyl, tetrahydropyranyl, morpholinyl or phenyl,
where cyclobutyl, cyclopentyl, cyclopentenyl, cyclohexyl, pyrrolidinyl, piperidinyl, tetrahydrofuranyl, tetrahydropyranyl, morpholinyl and phenyl may be substituted by 1 or 2 substituents independently of one another selected from the group consisting of fluorine, methyl, ethyl, trifluoromethyl, hydroxyl, methoxy and ethoxy,
and
L³ represents (C₁-C₃)-alkanediyl or a group of the formula •—W—CR⁹R¹⁰—••, •—W—CH₂—CR⁹R¹⁰—•• or •—CH₂—W—CR⁹R¹⁰—••,
where alkanediyl may be substituted by 1 or 2 fluorine substituents,
and
where
• represents the point of attachment to the ring Q,
•• represents the point of attachment to the group Z,
W represents O or N—R⁷,
where
R⁷ represents hydrogen, (C₁-C₃)-alkyl or cyclopropyl,
R⁹ represents hydrogen or fluorine,
and
R¹⁰ represents hydrogen or fluorine,
Z represents a group of the formula 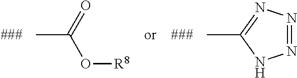
where
### represents the point of attachment to the group L¹ or L³,
and
R⁸ represents hydrogen,
R¹ represents fluorine, chlorine, methyl, ethyl, vinyl, trifluoromethyl or methoxy,
n represents the number 0, 1 or 2,
where, if R¹ is present more than once, its meaning may in each case be identical or different,
and
R² represents phenyl or 2-pyridyl,
where phenyl and 2-pyridyl may be substituted by 1 or 2 substituents independently of one another selected from the group consisting of fluorine, chlorine, cyano, methyl, ethyl, n-propyl, vinyl, methoxy, ethoxy, trifluoromethyl, trifluoromethoxy, methylthio, ethylthio, amino, methylamino and ethylamino,
and
R³ represents methyl or trifluoromethyl,
and salts thereof.

3. The compound of formula (I) as claimed in claim 1 in which
A represents —CH₂— or —C(═O)—,
E represents O or NR⁴,
where
R⁴ represents hydrogen,
M represents a group of the formula 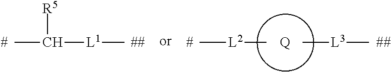
where
# represents the point of attachment to E,
## represents the point of attachment to Z,
R⁵ represents hydrogen or methyl,
L¹ represents butane-1,4-diyl, pentane-1,5-diyl or a group of the formula *-L^{1A}-V-L^{1B}-**,
where
* represents the point of attachment to —CHR⁵,
** represents the point of attachment to Z,
L^{1A} represents methylene or ethane-1,2-diyl,
where methylene and ethane-1,2-diyl may be substituted by 1 or 2 methyl substituents,
L^{1B} represents methylene or ethane-1,2-diyl,
and
V represents O or N—R⁶,
where
R⁶ represents methyl,
L² represents a bond,
Q represents phenyl,
and
L³ represents ethane-1,2-diyl, propane-1,3-diyl or a group of the formula •—W—CR⁹R¹⁰—•• or •—W—CH₂—CR⁹R¹⁰—••,
where
• represents the point of attachment to the ring Q,
•• represents the point of attachment to the group Z,
W represents O,
R⁹ represents hydrogen,
and
R¹⁰ represents hydrogen,
Z represents a group of the formula 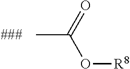
where
### represents the point of attachment to the group L¹ or L³,
and
R⁸ represents hydrogen,
R¹ represents fluorine, chlorine, methyl or trifluoromethyl,
n represents the number 0 or 1,
and
R² represents phenyl,
where phenyl may be substituted by a substituent selected from the group consisting of methyl, ethyl, vinyl, trifluoromethyl, methoxy, ethoxy and trifluoromethoxy,
and
R³ represents methyl,
and salts thereof.

4. A process for preparing compounds of formula (I) as defined in claim 1 in which Z represents —COOH, wherein either
[A] (1) a compound of the formula (II-A), 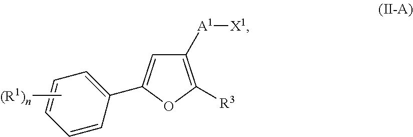
in which n, R¹ and R³ each have the meanings given in claim 1
and
A¹ represents —(C═O)—
and
X¹ represents chlorine or hydroxyl,
is coupled in an inert solvent, optionally in the presence of a suitable acid or base and/or a suitable condensing agent, with a compound of the formula (III-A)
HE-M-Z¹ (III-A),
in which E and M each have the meanings given in claim 1
and
Z¹ represents cyano or a group of the formula COOR^{8A},
where
R^{8A} represents (C₁-C₄)-alkyl,
to give a compound of the formula (IV-A) 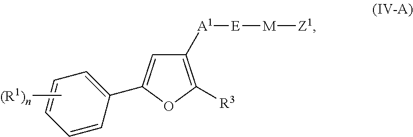
in which n, A¹, E, M, Z¹, R¹ and R³ each have the meanings given above,
(2) the compound of formula (IV-A) is brominated in an inert solvent to give a compound of the formula (V-A) 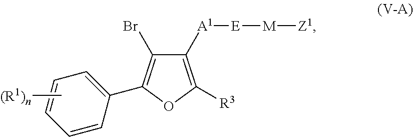
in which n, A¹, E, M, Z¹, R¹ and R³ each have the meanings given above,
and (3) the compound of formula (V-A) is coupled in an inert solvent in the presence of a base and a suitable palladium catalyst with a compound of the formula (VI) 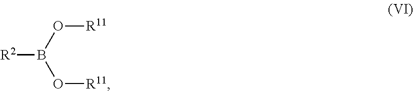
in which R² has the meaning given in claim 1
and
R¹¹ represents hydrogen or both radicals R¹¹ together form a —C(CH₃)₂—C(CH₃)₂— bridge,
to give a compound of the formula (VII-A) 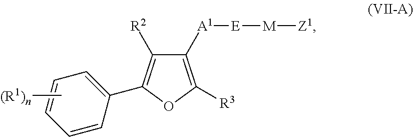
in which n, A¹, E, M, Z¹, R¹, R² and R³ each have the meanings given above,
or
[B] (1) a compound of the formula (II-B) 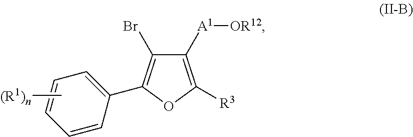
in which n, R¹ and R³ each have the meanings given in claim 1
and
A¹ represents —(C═O)—,
and
R¹² represents (C₁-C₄)-alkyl,
is coupled in an inert solvent in the presence of a base and a suitable palladium catalyst with a compound of the formula (VI) 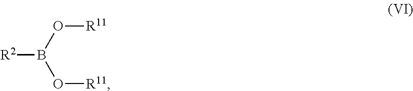
in which R² has the meaning given in claim 1
and
R¹¹ represents hydrogen or both radicals R¹¹ together form a —C(CH₃)₂—C(CH₃)₂— bridge,
to give a compound of the formula (IV-B) 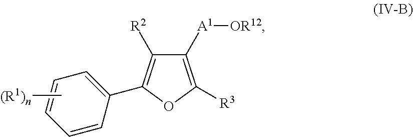
in which n, A¹, R¹, R², R³ and R¹² each have the meanings given above,
and (2) the compound of formula (IV-B) is converted by basic or acidic hydrolysis into a compound of the formula (V-B) 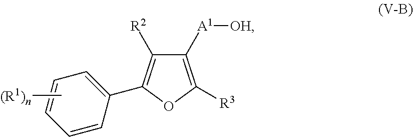
in which n, A¹, R¹, R² and R³ each have the meanings given above,
and (c) the compound of formula (V-B) is reacted in an inert solvent in the presence of a suitable base and a suitable condensing agent with a compound of the formula (III-A)
HE-M-Z¹ (III-A),
in which E and M each have the meanings given in claim 1
and
Z¹ represents cyano or a group of the formula COOR^{8A},
where
R^{8A} represents (C₁-C₄)-alkyl,
to give a compound of the formula (VII-A) 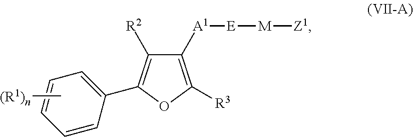
in which n, A¹, E, M, Z¹, R¹, R² and R³ each have the meanings given above,
or
[C] (1) a compound of the formula (II-C) 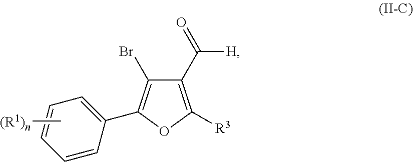
in which n, R¹ and R³ each have the meanings given in claim 1,
is coupled in an inert solvent in the presence of a base and a suitable palladium catalyst with a compound of the formula (VI) 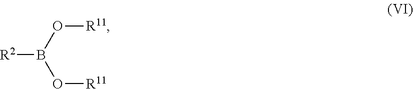
in which R² has the meaning given in claim 1
and
R¹¹ represents hydrogen or both radicals R¹¹ together form a —C(CH₃)₂—C(CH₃)₂— bridge,
to give a compound of the formula (IV-C) 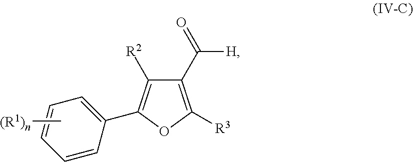
in which n, R¹, R² and R³ each have the meanings given above,
(2) the compound of formula (IV-C) is reduced in a suitable solvent with a suitable reducing agent to give a compound of the formula (V-C) 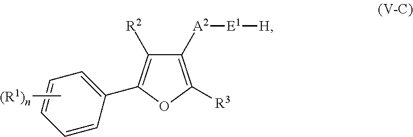
in which n, R¹, R² and R³ each have the meanings given above
and
A² represents —CH₂—
and
E¹ represents O,
(3) the compound of formula (V-C) is reacted in an inert solvent in the presence of a suitable base with a compound of the formula (III-C)
X²-M-Z¹ (III-C),
in which M has the meaning given in claim 1,
Z¹ represents cyano or a group of the formula COOR^{8A},
where
R^{8A} represents (C₁-C₄)-alkyl,
and
X² represents a leaving group,
to give a compound of the formula (VII-C) 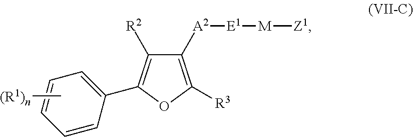
in which n, A², E¹, M, Z¹, R¹, R² and R³ each have the meanings given above,
or
[D] (1) a compound of the formula (IV-C) 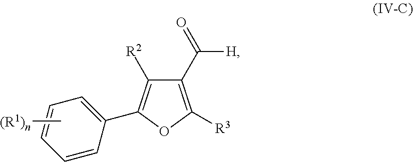
in which n, R¹, R² and R³ each have the meanings given in claim 1,
is reacted in an inert solvent in the presence of a suitable reducing agent with a compound of the formula (III-D)
HE²-M-Z¹ (III-D),
in which M has the meaning given in claim 1,
Z¹ represents cyano or a group of the formula COOR^{8A},
where
R^{8A} represents (C₁-C₄)-alkyl,
and
E² represents NR⁴,
where
R⁴ represents hydrogen or (C₁-C₄)-alkyl,
to give a compound of the formula (VII-D) 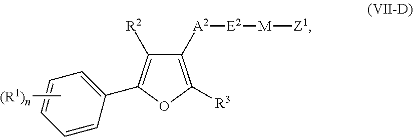
in which A² represents —CH₂— and n, E², M, Z¹, R¹, R² and R³ each have the meanings given above,
and the compound of formula (VII-A), (VII-C) or (VII-D) is converted by hydrolysis of the cyano or ester group Z¹ into a carboxylic acid of the formula (I-1) 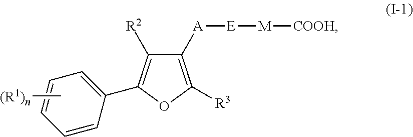
in which n, A, E, M, R¹, R² and R³ each have the meanings given above,
which is optionally reacted with the appropriate bases or acids to give a salt thereof.

5. (canceled)

6. (canceled)

7. (canceled)

8. A pharmaceutical composition comprising a compound of formula (I) as defined in claim 1 and an inert non-toxic pharmaceutically suitable auxiliary.

9. A pharmaceutical composition comprising a compound of formula (I) as defined in claim 1 and an additional active compound.

10. (canceled)

11. A method for the treatment and/or prophylaxis of angina pectoris, pulmonary hypertension, thromboembolic disorders and peripheral occlusive diseases comprising administering to a human or animal in need thereof an effective amount of at least one compound of the formula (I) as defined in claim 1.

12. A process for preparing compounds of formula (I) as defined in claim 2 in which Z represents —COOH, wherein either
[A] (1) a compound of the formula (II-A), 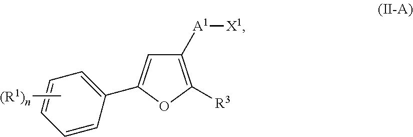
in which n, R¹ and R³ each have the meanings given in claim 2
and
A¹ represents —(C═O)—
and
X¹ represents chlorine or hydroxyl,
is coupled in an inert solvent, optionally in the presence of a suitable acid or base and/or a suitable condensing agent, with a compound of the formula (III-A)
HE-M-Z¹ (III-A),
in which E and M each have the meanings given in claim 2
and
Z¹ represents cyano or a group of the formula COOR^{8A},
where
R^{8A} represents (C₁-C₄)-alkyl,
to give a compound of the formula (IV-A) 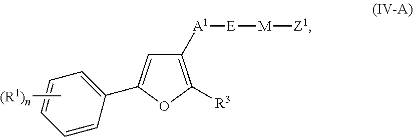
in which n, A¹, E, M, Z¹, R¹ and R³ each have the meanings given above,
(2) the compound of formula (IV-A) is brominated in an inert solvent to give a compound of the formula (V-A) 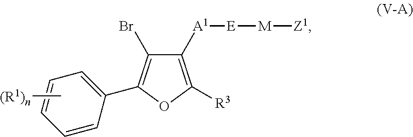
in which n, A¹, E, M, Z¹, R¹ and R³ each have the meanings given above,
and (3) the compound of formula (V-A) is coupled in an inert solvent in the presence of a base and a suitable palladium catalyst with a compound of the formula (VI) 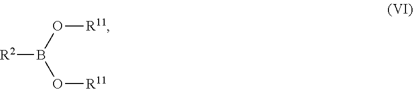
in which R² has the meaning given in claim 2
and
R¹¹ represents hydrogen or both radicals R¹¹ together form a —C(CH₃)₂—C(CH₃)₂— bridge,
to give a compound of the formula (VII-A) 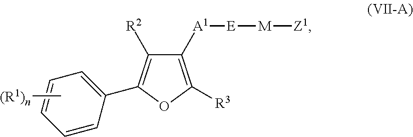
in which n, A¹, E, M, Z¹, R¹, R² and R³ each have the meanings given above,
or
[B] (1) a compound of the formula (II-B) 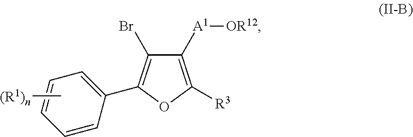
in which n, R¹ and R³ each have the meanings given in claim 2
and
A¹ represents —(C═O)—,
and
R¹² represents (C₁-C₄)-alkyl,
is coupled in an inert solvent in the presence of a base and a suitable palladium catalyst with a compound of the formula (VI) 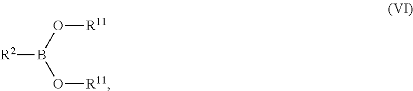
in which R² has the meaning given in claim 2
and
R¹¹ represents hydrogen or both radicals R¹¹ together form a —C(CH₃)₂—C(CH₃)₂— bridge,
to give a compound of the formula (IV-B) 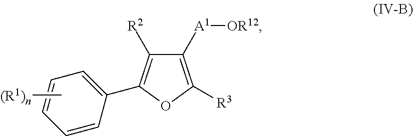
in which n, A¹, R¹, R², R³ and R¹² each have the meanings given above,
and (2) the compound of formula (IV-B) is converted by basic or acidic hydrolysis into a compound of the formula (V-B) 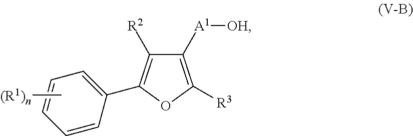
in which n, A¹, R¹, R² and R³ each have the meanings given above,
and (c) the compound of formula (V-B) is reacted in an inert solvent in the presence of a suitable base and a suitable condensing agent with a compound of the formula (III-A)
HE-M-Z¹ (III-A),
in which E and M each have the meanings given in claim 2
and
Z¹ represents cyano or a group of the formula COOR^{8A},
where
R^{8A} represents (C₁-C₄)-alkyl,
to give a compound of the formula (VII-A) 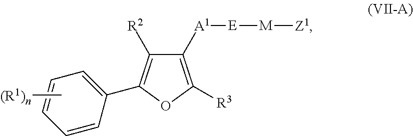
in which n, A¹, E, M, Z¹, R¹, R² and R³ each have the meanings given above,
or
[C] (1) a compound of the formula (II-C) 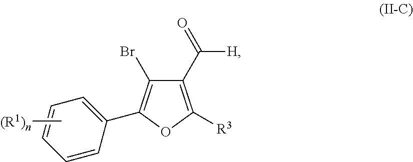
in which n, R¹ and R³ each have the meanings given in claim 2,
is coupled in an inert solvent in the presence of a base and a suitable palladium catalyst with a compound of the formula (VI) 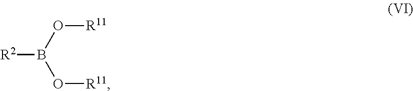
in which R² has the meaning given in claim 2
and
R¹¹ represents hydrogen or both radicals R¹¹ together form a —C(CH₃)₂—C(CH₃)₂— bridge,
to give a compound of the formula (IV-C) 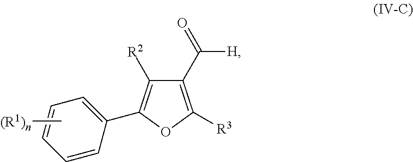
in which n, R¹, R² and R³ each have the meanings given above,
(2) the compound of formula (IV-C) is reduced in a suitable solvent with a suitable reducing agent to give a compound of the formula (V-C) 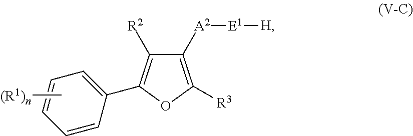
in which n, R¹, R² and R³ each have the meanings given above
and
A² represents —CH₂—
and
E¹ represents O,
(3) the compound of formula (V-C) is reacted in an inert solvent in the presence of a suitable base with a compound of the formula (III-C)
X²-M-Z¹ (III-C),
in which M has the meaning given in claim 2,
Z¹ represents cyano or a group of the formula COOR^{8A},
where
R^{8A} represents (C₁-C₄)-alkyl,
and
X² represents a leaving group,
to give a compound of the formula (VII-C) 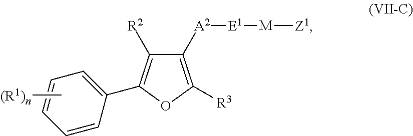
in which n, A², E¹, M, Z¹, R¹, R² and R³ each have the meanings given above,
or
[D] (1) a compound of the formula (IV-C) 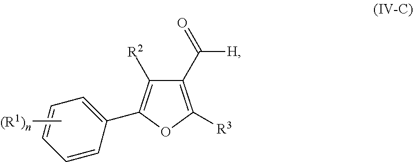
in which n, R¹, R² and R³ each have the meanings given in claim 2,
is reacted in an inert solvent in the presence of a suitable reducing agent with a compound of the formula (III-D)
HE²-M-Z¹ (III-D),
in which M has the meaning given in claim 2,
Z¹ represents cyano or a group of the formula COOR^{8A},
where
R^{8A} represents (C₁-C₄)-alkyl,
and
E² represents NR⁴,
where
R⁴ represents hydrogen or (C₁-C₄)-alkyl,
to give a compound of the formula (VII-D) 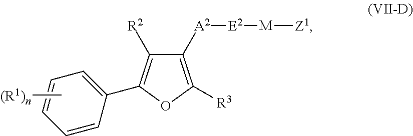
in which A² represents —CH₂— and n, E², M, Z¹, R¹, R² and R³ each have the meanings given above,
and the compound of formula (VII-A), (VII-C) or (VII-D) is converted by hydrolysis of the cyano or ester group Z¹ into a carboxylic acid of the formula (I-1) 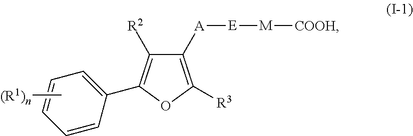
in which n, A, E, M, R¹, R² and R³ each have the meanings given above,
which is optionally reacted with the appropriate bases or acids to give a salt thereof.

13. A process for preparing compounds of formula (I) as defined in claim 3 in which Z represents —COOH, wherein either
[A] (1) a compound of the formula (II-A), 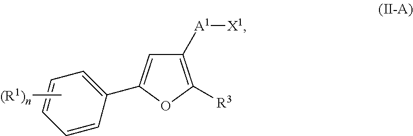
in which n, R¹ and R³ each have the meanings given in claim 3
and
A¹ represents —(C═O)—
and
X¹ represents chlorine or hydroxyl,
is coupled in an inert solvent, optionally in the presence of a suitable acid or base and/or a suitable condensing agent, with a compound of the formula (III-A)
HE-M-Z¹ (III-A),
in which E and M each have the meanings given in claim 3
and
Z¹ represents cyano or a group of the formula COOR^{8A},
where
R^{8A} represents (C₁-C₄)-alkyl,
to give a compound of the formula (IV-A) 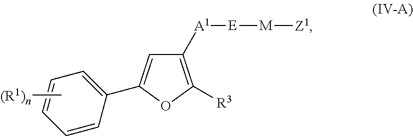
in which n, A¹, E, M, Z¹, R¹ and R³ each have the meanings given above,
(2) the compound of formula (IV-A) is brominated in an inert solvent to give a compound of the formula (V-A) 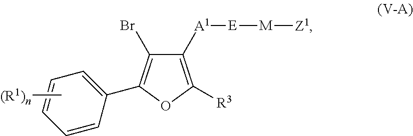
in which n, A¹, E, M, Z¹, R¹ and R³ each have the meanings given above,
and (3) the compound of formula (V-A) is coupled in an inert solvent in the presence of a base and a suitable palladium catalyst with a compound of the formula (VI) 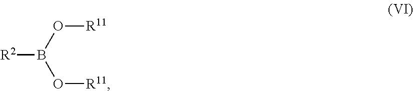
in which R² has the meaning given in claim 3
and
R¹¹ represents hydrogen or both radicals R¹¹ together form a —C(CH₃)₂—C(CH₃)₂— bridge,
to give a compound of the formula (VII-A) 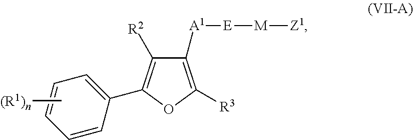
in which n, A¹, E, M, Z¹, R¹, R² and R³ each have the meanings given above,
or
[B] (1) a compound of the formula (II-B) 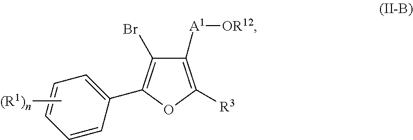
in which n, R¹ and R³ each have the meanings given in claim 3
and
A¹ represents —(C═O)—,
and
R¹² represents (C₁-C₄)-alkyl,
is coupled in an inert solvent in the presence of a base and a suitable palladium catalyst with a compound of the formula (VI) 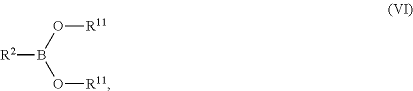
in which R² has the meaning given in claim 3
and
R¹¹ represents hydrogen or both radicals R¹¹ together form a —C(CH₃)₂—C(CH₃)₂— bridge,
to give a compound of the formula (IV-B) 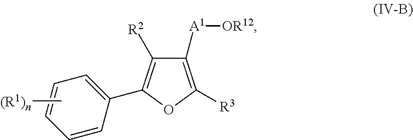
in which n, A¹, R¹, R², R³ and R¹² each have the meanings given above,
and (2) the compound of formula (IV-B) is converted by basic or acidic hydrolysis into a compound of the formula (V-B) 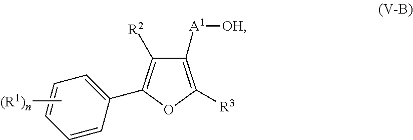
in which n, A¹, R¹, R² and R³ each have the meanings given above,
and (c) the compound of formula (V-B) is reacted in an inert solvent in the presence of a suitable base and a suitable condensing agent with a compound of the formula (III-A)
HE-M-Z¹ (III-A),
in which E and M each have the meanings given in claim 3
and
Z¹ represents cyano or a group of the formula COOR^{8A},
where
R^{8A} represents (C₁-C₄)-alkyl,
to give a compound of the formula (VII-A) 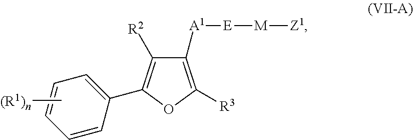
in which n, A¹, E, M, Z¹, R¹, R² and R³ each have the meanings given above,
or
[C] (1) a compound of the formula (II-C) 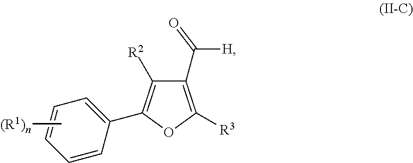
in which n, R¹ and R³ each have the meanings given in claim 3,
is coupled in an inert solvent in the presence of a base and a suitable palladium catalyst with a compound of the formula (VI) 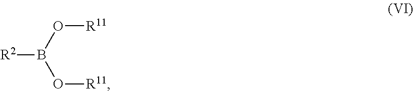
in which R² has the meaning given in claim 3
and
R¹¹ represents hydrogen or both radicals R¹¹ together form a —C(CH₃)₂—C(CH₃)₂— bridge,
to give a compound of the formula (IV-C) 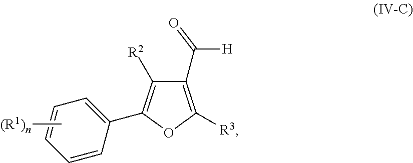
in which n, R¹, R² and R³ each have the meanings given above,
(2) the compound of formula (IV-C) is reduced in a suitable solvent with a suitable reducing agent to give a compound of the formula (V-C) 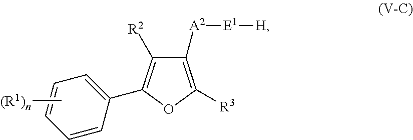
in which n, R¹, R² and R³ each have the meanings given above
and
A² represents —CH₂—
and
E¹ represents O,
(3) the compound of formula (V-C) is reacted in an inert solvent in the presence of a suitable base with a compound of the formula (III-C)
X²-M-Z¹ (III-C),
in which M has the meaning given in claim 1,
Z¹ represents cyano or a group of the formula COOR^{8A},
where
R^{8A} represents (C₁-C₄)-alkyl,
and
X² represents a leaving group,
to give a compound of the formula (VII-C) 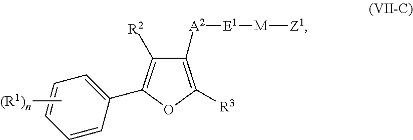
in which n, A², E¹, M, Z¹, R¹, R² and R³ each have the meanings given above,
or
[D] (1) a compound of the formula (IV-C) 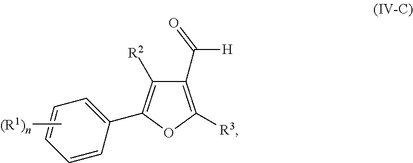
in which n, R¹, R² and R³ each have the meanings given in claim 3,
is reacted in an inert solvent in the presence of a suitable reducing agent with a compound of the formula (III-D)
HE²-M-Z¹ (III-D),
in which M has the meaning given in claim 1,
Z¹ represents cyano or a group of the formula COOR^{8A},
where
R^{8A} represents (C₁-C₄)-alkyl,
and
E² represents NR⁴,
where
R⁴ represents hydrogen or (C₁-C₄)-alkyl,
to give a compound of the formula (VII-D) 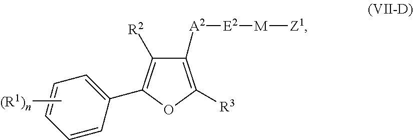
in which A² represents —CH₂— and n, E², M, Z¹, R¹, R² and R³ each have the meanings given above,
and the compound of formula (VII-A), (VII-C) or (VII-D) is converted by hydrolysis of the cyano or ester group Z¹ into a carboxylic acid of the formula (I-1) 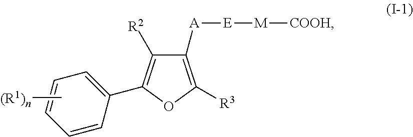
in which n, A, E, M, R¹, R² and R³ each have the meanings given above,
which is optionally reacted with the appropriate bases or acids to give a salt thereof.

14. A pharmaceutical composition comprising a compound of formula (I) as defined in claim 2 and an inert non-toxic pharmaceutically suitable auxiliary.

15. A pharmaceutical composition comprising a compound of formula (I) as defined in claim 3 and an inert non-toxic pharmaceutically suitable auxiliary.

16. A pharmaceutical composition comprising a compound of formula (I) as defined in claim 2 and an additional active compound.

17. A pharmaceutical composition comprising a compound of formula (I) as defined in claim 3 and an additional active compound.

18. A method for the treatment and/or prophylaxis of angina pectoris, pulmonary hypertension, thromboembolic disorders and peripheral occlusive diseases comprising administering to a human or animal in need thereof an effective amount of at least one compound of formula (I) as defined in claim 2.

19. A method for the treatment and/or prophylaxis of angina pectoris, pulmonary hypertension, thromboembolic disorders and peripheral occlusive diseases comprising administering to a human or animal in need thereof an effective amount of at least one compound of formula (I) as defined in claim 3.

20. A method for the treatment and/or prophylaxis of angina pectoris, pulmonary hypertension, thromboembolic disorders and peripheral occlusive diseases comprising administering to a human or animal in need thereof an effective amount of the pharmaceutical composition of claim 9.

21. A method for the treatment and/or prophylaxis of angina pectoris, pulmonary hypertension, thromboembolic disorders and peripheral occlusive diseases comprising administering to a human or animal in need thereof an effective amount of the pharmaceutical composition of claim 16.

22. A method for the treatment and/or prophylaxis of angina pectoris, pulmonary hypertension, thromboembolic disorders and peripheral occlusive diseases comprising administering to a human or animal in need thereof an effective amount of the pharmaceutical composition of claim 17.
